(19) United States
(12) Patent Application Publication
(10) Pub. No.: US 2025/0082809 A1
(43) Pub. Date: Mar. 13, 2025

(54) MALODOR CONTROL COMPOSITIONS AND THEIR USES

(71) Applicant: Kalykos LLC, Wilmington, DE (US)

(72) Inventor: Young Sam Kim, Daejeon (KR)

(73) Assignee: KALYKOS LLC, Wilmington, DE (US)

(21) Appl. No.: 18/579,104

(22) PCT Filed: Nov. 30, 2023
(86) PCT No.: PCT/US23/81759

§ 371 Date: Jan. 12, 2024

Related U.S. Application Data
(60) Provisional application No. 63/431,137, filed on Dec. 8, 2022.

(51) Int. Cl. A61L 9/012; A61K 8/02; A61K 8/19; A61L 9/014; A61L 101/02; A61Q 15/00

(52) U.S. Cl. CPC A61L 9/012; A61K 8/025; A61K 8/19; A61L 9/014; A61Q 15/00; A61L 2101/02

(57) ABSTRACT

The present invention relates to an activated composition comprising a spherical activated carbon particle of a sphericity greater than 0.6 and at least one of a plurality of possible active agents. The composition effectively sequesters the malodors, which includes complex fishy smell containing no amines, sulfur-containing malodors, and volatile organic compounds such as formaldehyde, volatile aldehydes, and ethylene. The activated composition of the present invention may be useful in consumer, personal care, home care, and healthcare-related products and industrial applications.

### FIELD OF THE INVENTION

The present invention relates to an activated composition for controlling unwanted odors, such as undesired malodors, including complex sulfur-containing malodors, and harmful VOCs (volatile organic compounds) odors, comprising a spherical activated carbon particle carrier.

### DESCRIPTION OF THE RELATED ART

There is a significant need to control malodors such as complex malodors of menstrual blood and vaginal discharge, sulfur-containing malodors, and VOCs (volatile organic compounds), including formaldehyde and other similar undesired odors, for use in various areas in consumer products and industrial applications. The state-of-the-art malodors and VOCs control agents are typically used in a fine powder, granular, or liquid form.

There are many patents and patent applications concerning malodor control agents, including for example, activated carbon powders, activated carbon granules, sodium bicarbonate (baking soda), zeolites, silicates, polymeric porous particles, and other odor control agents (e.g., U.S. Pat. No. 5,944,704A; WO2005120594A1; EP3321313B1; EP3564297B1, U.S. Ser. No. 10/702,626B2). Some commercial applications of these odor control agents focus on hygiene articles such as disposable baby diapers and adult incontinence products, light incontinence products, feminine napkins, panty liners, and deodorants for body odors and clothing, and other consumer products, where odor control is desired.

Inorganic fine powder agents such as zeolites and silicates are typically too abrasive and dusty to be used frequently in disposable consumer products.

Commercially preferred products use activated carbon (or active carbon) and sodium bicarbonate, primarily in a fine powder form, and are among the commercially most frequently used malodor control agents. One drawback to these agents is that handling and using very fine activated carbon and baking soda powder components in large quantities is cumbersome in manufacture and handling to make the final consumer products; and also may cause black color staining and cross-contamination.

Many methods known in the prior art disclose methods to introduce odor control properties into absorbent hygiene articles. Because of the dusty and fine powder nature of activated carbon powder (see FIG. 1), it has been typically used with a binder. For example, U.S. Pat. No. 5,407,442A and WO1991012030A1 use hydroxyethyl celluloses that are water-soluble polymers for the adhesion of activated carbon powder. Similar methods are disclosed in the following patents and patent applications:U.S. Pat. No. 8,552,251B2; JP6188035B2; WO2000004938A1; WO2009006377A1; among others.

As seen in this prior art, many methods use granular-type superabsorbent polymer (SAP) particles as carriers to introduce odor control properties into the absorbent articles. Still, these approaches have some critical drawbacks: the quantity of odor control agents attached to the SAP solid particles is limited due to their relatively large sizes and low surface areas available for binding. Typically, a low to medium one-digit percent by weight, usually below 5 percent by weight, of the odor control materials is used based on the total SAP weight, which is the preferred practical, usable quantity. This odor control agent's relatively low weight concentration, based on the total weight of the absorbent article, may not provide sufficient odor control efficacy in many cases. Even at such low concentrations, the process and/or handling of such odor control-treated SAP products still may experience significant issues, including but not limited to abrasion issues at processing equipment, dust, segregation during storage and transportation, and cross-contaminations due to insufficient bindings. Furthermore, the irregular and angular shapes of SAP particles may create pinholes in the thin back sheet films of the hygiene articles, which escalates the malodor issues.

Various methods have been tried to circumvent the dust issues of an activated carbon fine powder. For example, WO2006071313A1 and WO2004060421A1 disclose an odor-controlling, thin-coating layer comprising a viscose ink form comprising activated carbon, a binder, and a solvent, wherein the binder is a styrene polymer latex. However, such water, solvent, and dispersed solid polymer particle systems need drying, which may reduce the manufacturing efficiency of the products due to increased manufacturing time.

The abandoned US Pub Appln. US2012/0308507A1 discloses an odor-inhibiting mixture comprising water-absorbing polymer (SAP) particles and spherical activated carbon, wherein the spherical activated carbon particles are obtained from polymer pyrolysis of organic material, for example, polystyrene. However, despite the spherical nature of the polystyrene-based activated carbon particle, the SAP particles in the mixture are irregular-shaped granular particles, which may create issues of pinholes, abrasiveness, and attrition. Further, this US Pub. Appln. US2012/0308507A1 discloses the importance of SAP particle morphology and its impact on mechanical attrition. Therefore, odor control materials must possess robustness to withstand the potential mechanical attrition caused by the current granular SAP particle morphology due to irregular, angular, and edgy shapes.

U.S. Pat. No. 8,741,427B2 discloses a microcavity-containing resilient polyolefin-foam comprising a thermoplastic polymer containing diverse powder from odor control agents.

Most frequently, however, powdered active carbon and other odor control agents are adopted in matrices such as fibers, sheets, laminates, films, or a substrate containing the same, and foam, among others. Some of those patents and patent applications include JP2015070995A; U.S. Pat. No. 4,826,497A; JP2018166937A; JP2019170546A; US6245693B1; WO2009115931A2, WO1998028479A1; KR920000295A; DE10197025B3, among others.

However, apart from the cost related to the processing, the surfaces of the malodor and VOC control agents can get readily inactivated or become partly inaccessible when they are buried or partially embedded inside these matrices. In addition, the particles on the surfaces tend to shed and may create dust over time.

Recently, liquid formulations of malodors and VOCs abating products have gained tremendous popularity globally in consumer products. However, they often contain perfumes, fragrances, and other volatile solvents, which may unintentionally increase the VOC levels in closed spaces with insufficient air circulations or cause allergic reactions.

In addition, some popular liquid formulations contain cyclodextrins as a malodor control agent in addition to perfumes and fragrances. The perfumes and fragrances mask the malodors only and do not sequester the malodors. Cyclodextrins may cause stains on the surfaces when dried and help develop malodors themselves due to the bacterial metabolism of the sugar cyclodextrin, which may be accelerated in certain moist conditions.

Among the array of malodors and VOCs, sulfur-containing malodors present a significant challenge for masking with the technologies currently available: they are practically insoluble in water, chemically neutral, and notoriously pungent even at extremely low concentrations of ppm (parts per million), ppb (part per billion), and sometimes ppt (part per trillion) levels. Due to the high degree of the difficulties described above, little progress has been made to control these odors despite the enormous efforts made to solve the problem.

Therefore, what is needed is a control agent for malodors, sulfur-containing malodors, and VOCs, including formaldehyde, with easy handling, easy processing, no abrasion, no color staining, no cross-contamination, reduced dust formation of both manufacturing and the product, user-friendliness, and excellent efficacies.

### SUMMARY OF THE INVENTION

The present invention provides a composition comprising a spherical activated carbon particle having a sphericity greater than 0.6, and at least one active agent. These active agents are selected from a plurality of possible agents as discussed herein.

For example, an active agent can be used for controlling malodors and uses, therefore. These malodors include but are not limited to, any undesired aroma such as complex fishy smell containing no amines, sulfur-containing malodors, and volatile organic compounds (VOCs), including formaldehyde. The composition comprises spherical activated carbon particles having a sphericity greater than 0.6 and a plurality of active agents characterized by excellent effectiveness in controlling the malodors.

The present invention, in particular, provides an alleviation and control means for malodors associated with different sources. Some of these sources are found with complex fishy smells containing no amines, which is found in the complex malodors of menstruation blood, vaginal, and vulvar discharges; malodors associated with Kimchi malodors, which represent a complex pool of volatile sulfur-containing malodors; and malodors from various volatile aldehydes and VOCs, including formaldehyde and ethylene and others.

One example of a composition has: (a) about 5 to about 99.99 wt. % of spherical activated carbon particles comprising sphericity of at least 0.6, a particle diameter from about 0.01 to about 5000 μm, a BET-specific surface area of about 100 to about 2500 m²/g, an iodine adsorption capacity of about 100 to about 2500 mg/g and (b) about 0.01 wt. % to about 80 wt. % of at least one active agent, and (c) about 0.01 wt. % to about 10 wt. % water, based on the total weight of the composition. (These terms and measurements are defined in the glossary and later herein.)

The composition of the present invention represents a viable alternative, versatile and flexible, ease-to-process, and ease-to-use malodor control method, which is superior to the current existing state-of-the-art particulate materials such as activated carbon powder and sodium bicarbonate powder-type products.

The unique properties of the composition of the present invention include the highly spherical geometry, high surface areas, low dust and attrition, and high purity concerning ashes and heavy metals such as chromium. These are particularly beneficial characteristics for manufacturing various consumer product articles and developing new applications where such malodor control is desired. In addition, the composition of the present invention can be reactivated and recycled thanks to the robustness of the particles, which provides potential sustainability.

Additionally, the spherical geometry and high surfaces of the composition of the present invention provide an inherently constructive carrier to deliver and/or control the release of various active agents for diverse applications in an economical manner. These present particles are prepared using the process described in U.S. Pat. Nos. 3,909,449, 5,236,688, and 7,781,370.

When another moiety (compound, molecule, polymer) is added to the composition, it is an additive and forms an admixture with the composition and not an active agent of the composition. Thus, the active agent is a part of the composition by joining to the surface or entrapped or otherwise (for example, electrostatic or van der Waals surface forces) becomes a part of the spherical activated carbon particle; whereas the additive forms an admixture with the composition of this invention.

The composition of the present invention may be employed in extraordinarily diverse applications where odor control is desired or needed. Some of these uses include, but are not limited to: absorbent cores containing articles such as personal care hygiene articles, including disposable baby diapers and adult incontinences, light incontinences, feminine napkins, panty liners, tampons; pet hygiene products such as pet pads, pet diapers; health care and medical applications such as wound dressings, ostomy and colostomy bags, medical gowns and antiviral, antibacterial surgery masks, bed mat and sheets; other consumer related products such as oral care and smoke-free tobaccos such as nicotine pouches and snus; wine corks in bottles and barrels; food packages for Kimchi, Sauerkrauts, and cheeses; food packages for meats, fish, chickens, sausages, hams, and salamis; packages for ethylene emitting produce such as bananas, apples, avocados, melons, peaches, pears, tomatoes, plums, and cut-flowers such as carnations; cigarette filters for smoke cigarettes for removing ammonia and other VOCs, including formaldehyde and sulfur-containing compounds; antiviral and antibacterial facial masks; home care products such as garbage bags; medical and rescue uses such as body bags and blankets; household consumer products such as fridges, air filters, and air purifiers, among others.

### BRIEF DESCRIPTION OF DRAWINGS

The above and other features and advantages of the present invention will become more apparent when taken in conjunction with the following description and drawings wherein identical reference numerals have been used, where possible, to designate identical features that are common to the figures and wherein:

FIG. 1 is a photo of an activated carbon powder having no uniform particle size (Comparative Example A) disclosed herein.

FIG. 2 is a photo of sodium bicarbonate powder (baking soda) having no uniform particle size (Comparative Example B) disclosed herein.

FIG. 3 is a photo of the composition (Example 5) (spherical activated carbon particle based on grade G-BAC G-70R, e.g., derived from pitch raw materials) disclosed herein.

FIG. 4 is a photo of the composition (Example 7) (spherical activated carbon particle comprising citric acid based on grade G-BAC G-70R) disclosed herein.

### DETAILED DESCRIPTION OF THE INVENTION

For a better understanding of the principles of the present disclosure, reference to the embodiments of this invention will be discussed as illustrated in the specification and drawings.

It is understood that the terminology used herein is for the purpose of describing particular embodiments only and is not intended to be limiting. As used in this specification, the singular forms “a”, “an”, and “the” include plural referents unless the content clearly indicates otherwise. The following terms in the Glossary as used in this application are to be defined as stated below, and for these terms, the singular includes the plural.

Various headings are present to aid the reader but are not the exclusive location of all aspects of that referenced subject matter and are not to be construed as limiting the location of such discussion.

Also, certain US patents and PCT published applications have been incorporated by reference. However, the text of such patents is only incorporated by reference to the extent that no conflict exists between such text and other statements set forth herein. In the event of such conflict, then any such conflicting text in such incorporated by reference US patent or PCT application is specifically not so incorporated in this patent.

### Glossary

A-BAC-MP means spherical activated carbon particles from Kureha Corporation, Japan, having an average particle size and the weight fraction of 0.50±0.05 mm, 0.25 mm or less ≤5 wt. %, 0.71 mm or more ≤10 wt. %.

Acid compound is a carboxylic acid compound and includes acetic acid, ascorbic acid, formic acid, citric acid, fumaric acid, gluconic acid, methyl fumaric acid, malic acid, maleic acid, methyl maleic acid, itaconic acid, succinic acid, 2-methyl succinic acid, lactic acid, salicylic acid, powder of apple cider vinegar, powdered vinegar, or a combination thereof. (A. D. Pomogailo et al., Monomeric and Polymeric carboxylic acids, Chapter 2 in Macromolecular Metal Carboxylates and Their Nanogranules, Springer Series in Materials Science 138, Springer-Verlag Berlin Heidelberg 2010). Desirable carboxylic acids are citric acid, and tartaric acid from about 0.1 wt. % to 80 wt. % based on the total acid-activated composition weight. Carboxylic acid is a well-known biomaterial with various advantages, including biodegradability.

Acid-treated spherical activated carbon means an acid-activated composition (including citric acid-activated composition), acid-treated activated carbon particle, activated composition comprising acid, acid surface treated activated carbon particle, acid-coated spherical activated carbon particles, and acid-encapsulated activated composition.

Base compound includes sodium carbonate, sodium bicarbonate (baking soda), sodium silicate (water glass), sodium hydroxide, potassium hydroxide, ammonium hydroxide, calcium hydroxide, sodium monoxide, calcium oxide, aluminum hydroxide, cesium hydroxide, magnesium hydroxide, barium hydroxide, ferric hydroxide, and pyridine, and quaternary ammonium hydroxide, or a combination thereof.

Carbon tetrachloride activity measures the loading of carbon tetrachloride and weight percent on carbon (JIS K1474-5.1.2. Standard) The method measures the pore volume of the activated carbon and is primarily used as a quality assurance test for producing activated carbon.

Composition or activated composition means the composition as defined for this invention.

Control or controlling means combating, sequestrating, reducing, or removing.

G-BAC GR-70R means spherical activated carbon particles from Kureha Corporation, Japan, having an average particle size and the weight fraction of size of 0.70 mm ≤, 5, 0.6 mm or less ≤5 wt. %.

GRAS means Generally Recognized As Safe under §§ 201(s) and 409 of the Federal Food, Drug, and Cosmetic Act (US FDA); that is, any substance that is intentionally added to food is considered a food additive that is subject to premarket review and approval by US FDA unless the substance has GRAS certification.

Iodine adsorption number (in mg/g of carbon) measures the amount of iodine that can be adsorbed on the surface of a given mass of carbon black. (ASTM D1510-21) The iodine adsorption number depends on the activated carbon's micropore (0-2 nm) content by adsorption of iodine from the solution. (C. Saka, “BET, TG-DTG, FT-IR, SEM, iodine number analysis and preparation of activated carbon from acorn shell by chemical activation with ZnCl2,” Journal of Analytical and Applied Pyrolysis, 95, pp. 21-24, (2012)).

Krumbein chart for visual determination of roundness shows examples of pebbles for which the roundness has been grouped into nine classes. Although the Krumbein chart is a 2D instead of a 3D method, it clearly shows the differences between various sphericities. (W. C. Krumbein, “Measurement and geological significance of shape and roundness of sedimentary particles”, J. Sedimen. Res. 11 pp 64-72 (1941)) The Krumbein chart is adopted in the present invention to define sphericity. Therefore, any numerical definition of the sphericity used in the present invention should be referred to the Krumbein chart.

PureCarbon L means non-treated activated carbon particles derived from polymer beads, i.e., ion exchange beads, obtained from PureSphere Co., LTD, South Korea

Malodors means any undesired aroma, acidic odor compounds, basic odor compounds, sulfur-containing malodors, and volatile organic compounds (VOCs), including formaldehyde and ethylene gases.

Malodor control means the result of removal or lessening of malodors by use of the present malodor composition.

MCT means medium-chain triglyceride.

Melting temperature depression of a phase change material (PCM), such as wax, means melting point depression.

PCM means phase change materials, which are reservoir materials for thermal energy as latent heat and described further herein.

Pitch means a viscoelastic material that is composed of aromatic hydrocarbons. Pitch is produced via the distillation of carbon-based materials, such as plants, crude oil, and coal.

Powder means finely divided particles that are very small relative to the dimensions of the surface to which the powder is applied Typically, the inorganic or organic powders used in the method of this invention will have a particle size sufficient to pass through a sieve having openings of 45 microns or less.

Room temperature means an ambient temperature of about 20-25° C.

Saliva-permeable pouch or Sachet means an oral use where saliva can access the contents of the pouch.

Sniff testing is performed to assign scores according to the reduction of malodors of the spherical activated carbon adsorbent to be tested; the words sniff test, malodor test, odor control test, and olfactory test can be used interchangeably.

Sodium silicates are usually supplied as a viscous aqueous solution. Soluble sodium silicates, also known as “water-glass,” are colorless inorganic materials that are generally considered non-toxic and environmentally harmless. In addition, sodium silicates, as water-borne liquid silicates, are easy to handle, versatile, advantageous at low cost, and are available commercially.

Sphericity is a measure of how spherical an object is. For example, as proposed by Wadell defined in 1935, the sphericity, Ψ, of a particle is the ratio of the surface area of a sphere with the same volume as the given particle to the surface area of the particle (Wikipedia.org/wiki/sphericity):

$$Ψ = [ {{π}^{1 / 3} ( 6 {V}_{p} )}^{2 / 3} ] / {A}_{p} ,$$

where Vₚ is the volume of the particle and Ap is the particle's surface area. This test measures how closely the shape of a particle resembles that of a perfect sphere. Any particle that deviates from a sphere will have a sphericity less than 1. Some typical sphericities of practical samples are as follows: crushed glass 0.65; crushed coal 0.75; common salt 0.84; crushed sandstone 0.8-0.9; and round sand 0.92-0.98. (D. Wang, L.-S. Fan in Fluidized Bed Technologies for Near-Zero Emission Combustion and Gasification. A volume in Woodhead Publishing Series in Energy, 2013).

Spherical activated carbon particle means the non-treated and unprocessed spherical activated carbon particle, which may be subjected to further treatment with diverse, active agents; and includes particles derived from pitch, non-treated spherical activated composition derived from ion exchange beads or cellulose, or non-treated spherical activated carbon particles from granulation technologies.

Spherical cellulose particles or SCP mean cellulose beads or cellulose spheres, (e.g., Cellets™ from Transpharma Sanaq AG (Basel, Switzerland) as described further herein.

The ratio d(90) to d(10) qualitatively characterizes the distribution width, wherein d(10) and d(90) are percentile values that can be read directly from the cumulative particle size distribution. d(10) means 10% of the total particles are smaller than this diameter; d(90) represents 90% of the total particles are smaller than this diameter. d50 is also known as the median diameter. The wider the distribution, the larger the standard deviation.

Treatment of spherical activated carbon particles means surface treating and/or surface coating of the particles unless it is stated particles are immersed in a solution. Treatment of spherical activated carbon particles and their composition typically was conducted by mixing the particles with the solutions or melting such as wax under agitation.

VOC means volatile organic compounds, particularly those having undesired odors such as formaldehyde, ethylene, hexane, methyl pyrrole, pyridine and others described herein.

Water-soluble polymers and copolymers used in the present invention represent a diverse class of macromolecules, including naturally occurring polymers such as polysaccharides, biopolymers such as proteins and polypeptides, and various types of synthetic and semi-synthetic homopolymers, copolymers and block copolymers. (See “Water Soluble Polymers,” Encyclopedia of Polymer Science and Technology, John Wiley & Sons, Inc., Eds. Charles L. McCormick, Andrew B. Lowe, Neil Ayres) Preferred naturally occurring water-soluble polymers include, for example, one or more selected from the group consisting of biodegradable natural water-soluble polymers such as konjac glucomannan; sodium alginate; guar gum; potato starch, wheat starch, corn starch; pectin; agar-agar; carrageenan; konjac and carrageenan; gellan gum; gelatin; poly(lactic acid) (PLA), or one or more of a carboxylate and/or aldehyde and/or hydroxyl group includes one or more selected from the group consisting of a biodegradable semi-synthetic water-soluble polymer such as Methocel, and Hydroxypropyl cellulose (commercially available from The Dow Chemical Company), or a mixture thereof.

### Process for Making a Spherical Activated Carbon Particle and an Active Agent

A method of preparing the present composition comprises the following steps:
(A) Optionally, using activated spherical carbon particles comprising carbon from about 90 wt. % to 99.99 wt. % and water from about 0.01 wt. % to 10 wt. % based on the total weight of the composition; and
(B) preforming at least one of the following:
   (a) Preparing an aqueous solution comprising one of the actives, including an acid, a base, or urea, or mixtures thereof, to form a homogenous mixture; or
   (b) Optionally, preparing an aqueous solution comprising one or more of the active release agents selected from the group of a bioactive agent; surface active agent; medicinally and/or hygienically active agent; cosmetically active agent; stimulating agents such as nicotine and its salts, caffeine, and cannabinoids; essential oil, flavoring agent, sweetening agent, or a combination thereof; optionally, homogenizing and aging this aqueous solution; and
   (c) Applying independently one or more active release agents by spraying, immersion, blending, or a combination thereof to treat the activated composition; and
   (d) Drying the wet-state activated composition at an elevated temperature from about room temperature to 180° C. for 1 minute to 180 minutes by employing an air convection oven dryer, belt dryer, drum dryer, fluidized bed dryer, microwave dryer, infrared dryer, UV dryer, or a combination thereof to form a dry-state activated composition; or
   (e) Optionally, applying molten wax or aqueous wax dispersion by employing spraying mixing; and
(C) Sieving; or
(D) Optionally, (a) mixing the activated composition with particulate auxiliary odor control agents; and (b) adding a colloidal particle to the activated composition particle; wherein the activated composition comprises:
   (a) about 5 wt. % to 99.99 wt. % of spherical activated carbon particles comprising a sphericity of at least 0.6, a particle diameter in the range of about 0.01 μm to about 5000 μm, a BET-specific surface area of from about 100 to 2500 m²/g, or further an iodine adsorption capacity of 500 mg/g or greater;
   (b) about 0.01 wt. % to about 10 wt. % of spherical activated carbon particles comprising water;
   (c) about 0.1 wt. % to about 80 wt. % of a treatment compound of an acid, base, or urea, or mixtures thereof;
   (d) about 0.01 wt. % to about 15 wt. % of a colloidal particle;
   (e) about 0.01 wt. % to about 80 wt. % of a powder-type auxiliary odor control agent;
   (f) about 0.001 wt. % to about 5 wt. % for a soluble metal salt-type auxiliary odor control agent;
   (g) about 0.01 wt. % to about 25 wt. % of one or more active agents selected from the group of bioactive agents, essential oils, flavoring agents, sweetening agents, surface active agents, medicinally and/or hygienically active agents, cosmetically active agents, or stimulating agents such as caffeine and its salts, nicotine, and its salts, and/or cannabinoids oils and its salts; and
   (h) about 0.01 wt. % to about 80 wt. % of phase change material (PCM); wherein the wt. % is based on the total activated composition weight and with the proviso that the sum of the percentages of (a) to (h) equals 100 percent.

### Uses or Applications of Spherical Activated Carbon Particle Compositions

Because of the various sources of malodors and the environments in which they can happen, it is believed that so long as these present compositions can be utilized in the manufacture of such products to control malodors or be exposed to the malodor, they will be effective in lessening their effects. Therefore, this listing could be very broad and encompass many uses, some of which are provided below, but such listing is not to be limited or all-inclusive in indicating of listing all such applications. Generally, the active agent as a part of the composition is selected to control the intended malodor from any undesired aroma, sulfur-containing malodors, and VOC compounds, complex fishy smells, and complex sulfur-containing malodors (e.g., Kimchi malodors), including volatile acid malodors, volatile aldehydes such as formaldehyde, or VOC such as ethylene gases. The following listing is examples of, but not limited to, such uses and applications:

Complex malodors of menstrual blood and vaginal discharge Such odors arise from body fluids from blood, vaginal discharges, and the cells and fluid of the late secretory phase of the uterine endometrial lining that is shed during menstruation. These odors can be smelled as a fishy smell, a rotten smell, a metal-like smell, a potent blood smell, a sweaty smell, and a garlic- or onion-like smell, among others. Therefore, under normal menstrual conditions, wherein the proteins are present in the vaginal discharges and oils in the form of fatty acids on the skin in the vulva areas, the complex menstrual malodors are believed to be a complex mix of volatile organic compounds comprising aldehydes and ketones, and organophosphorus compounds in addition to the volatile sulfur-containing malodors. Therefore, reducing these malodors is desired and can be better achieved by incorporation of the present composition into various feminine hygiene products.

Complex Fishy Smells. A fishy smell is commonly associated with amine and ammonia, which frequently occur in hygiene applications such as feminine napkins, adult incontinence diapers, and light incontinence diapers, among others. Typically, the fishy smells of urines in adult and light incontinence diaper applications are attributed to food digestion: such digestive fishy smell is primarily attributed to amines, which can be found in raw fish smells. In the art, the fishy smell has been simulated typically by amine such as trimethyl amine (TMA). Further, such TMA-type of amine malodors have been circumvented by using acids such as citric acid in the art based on the acid and base reaction.

However, the fishy smell of women's menstruation blood and vaginal discharges is far more complex (therefore termed a “complex fishy smell”) and cannot be simply attributed to simple amines from food digestion. The ramifications have been it was challenging to test and evaluate the performances of new materials due to a lack of reliable complex fishy smell.

In the present invention, surprisingly, it was found that commercially available marine omega-3 oil or omega-3 fatty acid (termed hereafter marine omega-3 oil to differentiate it from the plant-based omega-3 oils), also called fish oil, suffice these testing requirements. Therefore, the commercially available marine omega-3 oil is a reliable source for complex fishy smell malodors that is not comprising amines and thus provides a unique and effective testing method for the odor control performance for various products such as feminine napkins, tampons, panty liners, light incontinence diapers, adult incontinence diapers, and wound dressings, among others.

Kimchi Smell as a Complex Pool of Volatile Acid- and Sulfur-Containing Malodors. Kimchi is a traditional Korean side dish composed of leaf and root vegetables with seasonings such as garlic, ginger, onion, red pepper powder, green onion, fermented shrimp, and anchovies. Recently, it has gained popularity as a functional food because of its touted anticancer activity, antitumor effects, and health-promoting effects, including improved fasting blood glucose, serum lipid profiles, and total antioxidant status.

However, when Kimchi is well-aged, microbiological and enzymatic activities due to the naturally occurring lactic acid bacteria and yeasts in the raw materials lead to a sour and bitter taste, CO₂ production, and malodors that emit unique malodors and become sour due to the increased number of free acids, which increases with fermentation. Among Kimchi's volatile organic acids, acetic acid and lactic acid are identified as significant acids. Leuconostoc mesenteroides produce the former and Lactobacillus brevis and the latter by lactic acid bacteria, respectively. Approximately 40 volatile ingredients have been identified in Kimchi. Volatile compounds such as allyl mercaptan, methyl allyl sulfide, dimethyl disulfide, diallyl sulfide, methyl trisulfide, and diallyl disulfide are mainly responsible for the odor associated with the fermentation of Kimchi. These compounds are assumed to be produced from their precursors, such as sulfoxides, thioglucosides, sulfur-containing amino acids, and sulfonium. Especially the smell of Kimchi, caused by volatile organic compounds (VOCs) with functional groups such as sulfur-containing and acidic, is a well-known undesirable characteristic. (Korean Journal of Packaging Science & Technology Vol. 26, No. 1, 1˜9 (2020), S. R. Yoo et al., “Evaluation of Deodorization Capabilities, Morphologies, and Thermal Stabilities of Baking Soda, Charcoal, Coffee, and Green Tea for Kimchi Packaging Application”).

Sulfur-containing Malodors. Due to the abundant presence of sulfurs in foods, amino acids, and proteins, their metabolites from the human body, digestions, and/or microbial metabolisms, the sulfur-containing malodors are ubiquitous. The following malodors areas show some potential applications of the composition of the present invention, wherein the sulfur-containing malodors are challenging and are a small cross-section of the enormous numbers of examples.

Wine Malodors. Malodors recognized in wine include those produced by butanoic acid (rancid butter) and propanoic acid (goaty smell), 3-methyl-2,4-nonanedione (the rancid character of old oxidized red wines), malodors of rubbery (like a thiol), and earthy (one or more sesquiterpenes). (R. S. Jackson, Chapter 3—Olfactory Sensations, Wine Tasting 3^{rd} Ed., A Professional Handbook 2017, Pages 41-101, Academic Press).

Mouth Malodors. The mouth is home to hundreds of bacterial species that produce several fetid substances from metabolism, such as protein degradation. Sulfur-containing malodorous compounds are produced by bacteria such as Porphyromonas, Prevotella, Actinobacillus, and Fusobacterium species. These are the most common organisms in the mouth, which have recently been identified as partly responsible for Halitosis, wherein methyl mercaptan and hydrogen sulfite were malodors. (Quantitative detection of the volatile sulfur compound-producing microorganisms in oral specimens using real-time PCR; H. Kato et al., pages 67-71, volume 11, issues 1, March 2005, J. of Oral Diseases).

Wound Dressing and Malodors. In wounds, malodor is the byproduct of bacteria and fatty acids that are part of necrotic tissue and harms the patient's quality of life. An unpleasant odor is often found in chronic wounds, especially those not closed after three months of treatment. (A. Akhmetova et al., in. J. Wound Ostomy Continence Nurs. 2016; 43(6): 598-609).

Ostomy/Colostomy Bag and Malodors. A colostomy bag is a plastic waterproof bag that collects fecal matter from the digestive tract through an opening in the abdominal wall called a stoma. An opening between the large intestine and the abdominal wall is formed during a colostomy. Odors from such a bag are well known.

Antiflatulence for Volatile Sulfur-containing Compounds. Everyone releases gas through belching (burping) or flatulence (farting). Some farting is normal, but excessive farting is often a severe problem that might be caused by diseases of chronic intestinal conditions, such as diverticulitis, ulcerative colitis, Crohn's disease, or a digestive system disorder, such as irritable bowel syndrome (IBS). In addition, an overgrowth or sudden change in the bacteria populations in the small intestine may also cause excess gas. When these vapors mix with intestinal bacteria, unpleasant volatile sulfur-containing malodors can develop, and excessive flatulence with foul-smelling malodors can be a serious privacy issue. Therefore, an effective method to reduce the malodors from flatulence is needed.

Washable (Reusable) Menstrual Underwear and Malodors. Most recently, washable (reusable) menstrual underwear (also called period underwear or menstrual panties) and incontinence pants have gained popularity among young adults. The period panties are eco-friendly and inexpensive despite the higher cost at the beginning due to the more extended usage. Typically, period underwear comprises elastic fabric made of textile and soft cotton for touching skin areas to give a dry feel. An internal layer of waterproof but breathable textile material is placed inside the period underwear to act as a barrier and help avoid stains. In addition, elastic fabrics are typically odor-controlling fabrics comprising antibacterial textiles.

The absorbent layer in the period underwear consists of mostly fixed thicknesses. Depending on the flow amount, the washable period underwear can be combined with a menstrual cup or tampons to retain more. However, some recent models comprise adjustable and detachable absorbent textile layers which can be placed in the open pouch in the menstrual panty. (www.teby.it). The period panties are washed by soaking in cold water or running cold water, which helps remove blood stains, and rubbing with soap by hand or in the washing machine at about 40 to about 50° C., followed by drying in the air.

Open-Cell Foams. The term foam means a structure comprised primarily of a thermoplastic polymer such as polyurethane and polyolefin materials having an open or closed cellular structure that does not collapse. The open-cell structured foam is preferred, and the term open-cell foam means a foam having an open cell content of at least 80% or greater, as determined by and according to ASTM D2856A.

The present composition can be integrated into a thin foam in period underwear. A soft and flexible polymer such as open-cell polyurethane foam is preferred. Also, cellulose-based elastic foams can be prepared using nanocrystalline cellulose (NCC), which is ultralight, elastic, and renewable. The composition of the present invention can be sandwiched between two thin layers of such soft open-cell foams and sealed by heat-sealing and/or applying micro dots of adhesives.

Footwear Odors. It is well known that all conventional shoes, for example, leather shoes, boots, work shoes, and sneakers, can generate bad smells. The primary reason is that modern shoes are constructed to cover the outside of the foot hermetically, while the foot sweats and creates high humidity microenvironment in the shoe for a prolonged time, causing severe foot odor and an athlete's foot. The foot malodors in the shoe interior space are volatile organic compounds (VOCs), identified as straight- and branched C₆-C₁₀ chains, volatile organic acids. (Caroprese A, Gabbanini S, Beltramini C, Lucchi E, Valgimigli L (2009) HS-SPME-GC-MS analysis of body odor to test the efficacy of foot deodorant formulations. Skin Res and Tech 15:503-510).

Formaldehyde. Although formaldehyde is not malodorous, its toxicity is exceptionally high, wherein the WHO has established a guideline level of 0.08 ppm. Exposure to formaldehyde as a harmful VOC is a problem There are small amounts of formaldehyde in nearly all homes. Formaldehyde is used in many common items, such as in pressed-wood products; glues and adhesives; permanent-press fabrics; paper product coatings; and certain insulation materials, including urea formaldehyde foam insulation (UFFI), and household products like flooring, furniture, and fabric. Formaldehyde has long been associated with sick building syndrome. (www.atsdr.cdc.gov/formaldehyde). The levels reduce over time. Most formaldehyde is released within two years (J. Park and R. Ikeda, 2006. “Variations of formaldehyde and VOC levels during three years in new and older homes.” Indoor Air. 16:129-135). Formaldehyde is a colorless, flammable gas at room temperature and has a strong odor. Exposure to formaldehyde may cause adverse health effects. (www.epa.gov/formaldehyde). There have been no effective technical solutions to keep or significantly lower the formaldehyde levels

### Further Additives to the Present Composition

Auxiliary Odor Control Agent to Use with the Present Composition

The composition of the present invention may contain an auxiliary odor control agent that may include one or more of the additives that form an admixture selected from the group comprising spherical cellulose particles as carriers treated for odor control; powdered, granulated, extruded activated carbon, or spheronized activated carbon composition (spherical activated carbon particles from granulation technologies); a cyclodextrin; a porous polymer adsorbent; an ion exchange polymer; natural and synthetic zeolites; a zeolite powder ion-exchanged with silver, copper, and/or zinc ions; chitosan; a plant extract as a dry powder of one or more green tea leaves, olive leaves, Yucca, Aloe, and Quillaja; or may include one or more of the water-soluble or readily dispersible actives selected from the group comprising borax; a peroxide such as sodium, potassium or ammonium percarbonate, peroxide and peroxodisulfate a permanganate such as sodium or potassium permanganate; a biocide; chelating agents; perfumes; various metals and metal compounds, including copper sulfate, copper acetate, zinc sulfate, zinc chloride, zinc ricinoleate, colloidal silver nano-particles, silver acetate, silver nitrate, and silver thiosulfate complexes, or a mixture thereof and used in a concentration of from about 0.01 wt. % to about 80 wt. % of a powder type auxiliary odor control agent; and about 0.001 wt. % to about 5 wt % for a soluble metal salt-type auxiliary odor control agent based on the weight of the total active composition.

### Colloidal Particles

The composition of the present invention may contain inorganic and/or organic colloidal particles and fine-sized micron-sized or nano-sized powders of silica, such as SiO₂, for example, fumed silica (hydrophobic and hydrophilic Aerosil brands from Degussa), colloidal silica (Ludox brands from Grace Davidson), and precipitated silica (Ultrasil brands from Degussa); alumina, titania, zirconia, diatomaceous Earth; superhydrophobic Aerogels such as silica, carbon, or cellulose aerogels, metal aerogel powders; cellulose nanofibers; cellulose nanofibers, or mixtures thereof. These colloidal particles are used in the concentration of about 0.001 wt % to about 15 wt. % based on the total activated composition weight. These may be used with the composition. They are not active agents.

### Active Release Agents

The present invention's composition may comprise as an active agent, a moiety that is released during use. The active release agent is dispersed and/or dissolved in an aqueous alcoholic solution before use. The active release agent includes an antibacterial metal ion such as zinc (2+) ion and copper (1+) ion, an ionic silver in various forms; a metallic colloidal silver, a water-soluble polymer gels such as Aloe vera; an anti-viral agent; an antimicrobial agent; an antioxidant; an anti-inflammatory agent such as an extract of magnolia or clove; an enzyme; a probiotic; a prebiotic; a dental agent such as a whitening agent, a desensitizing agent, a breath freshening agent, a mouth malodor preventing agent, chlorhexidine; nicotine and its salt; caffeine, cannabinoids, or a mixture thereof. These are active agents of the composition.

An example of the present composition comprises:
(a) about 5 wt. % to about 99.99 wt. % of spherical activated carbon particles comprising carbon derived from pitch or ion exchange beads having sphericity of at least 0.6, a particle diameter of about 0.01 μm to about 5000 μm, a BET-specific surface area of from about 100 to about 2500 m²/g, and an iodine adsorption capacity of 500 mg/g or greater;
(b) about 0.01 wt. % to about 10 wt. % of water;
(c) about 0.1 wt. % to about 80 wt. % of a treatment compound of an acid, base, or urea, or mixtures thereof;
(d) about 0.01 wt. % to about 15 wt. % of a colloidal particle;
(e) about 0.01 wt. % to about 80 wt % of a powder-type auxiliary odor control agent;
(f) about 0.001 wt. % to about 5 wt. % for a soluble metal salt-type auxiliary odor control agent; and
(g) about 0.001 wt. % to about 80 wt. % of one or more active agents that are release agents selected from bioactive agent, surface active agent, medicinally and/or hygienically active agent, cosmetically active agent, or stimulating agents such as, nicotine and its salts, caffeine, cannabinoids; essential oil; flavoring agent; or sweetening agent; wherein the wt. % is based on the total activated composition weight, and
with the proviso that the sum of the percentages of (a) to (f) equals 100 percent.

### Hyaluronic Acid and Botulinum Toxin.

Hyaluronic acid is a polymer of disaccharides composed of D-glucuronic acid and N-acetyl-D-glucosamine, linked via alternating β-(1→4) and β-(1→3) glycosidic bonds. Hyaluronic acid polymers can range from 5,000 to 20 million Da in vivo. The average molecular weight in human synovial fluid is 3-4 Million Da. Hyaluronic acid can help increase skin moisture and reduce the appearance of fine lines and wrinkles. Topical treatments can soothe redness and dermatitis, while injections can make skin appear firmer; botulinum toxin, often called Botox, is a neurotoxic protein produced by the bacterium Clostridium botulium and related species. The toxin is used for medical and cosmetic purposes. (www.Wikipedia/Botox). In cosmetic applications, botulinum toxin is considered relatively safe and widely used to reduce facial wrinkles. Injection of botulinum toxin into the muscles under facial wrinkles causes relaxation of those muscles, resulting in the smoothing of the overlying skin. Muscles can be treated repeatedly to maintain a smoother appearance. These are active agents of the composition.

### Alpha Hydroxy Acids (AHAs)

Alpha hydroxy acids (AHAs) consist of a carboxylic acid with a hydroxyl group substituent on the adjacent (alpha) carbon. Examples are glycolic acid, lactic acid, mandelic acid, and citric acid. AHAs are generally safe when used on the skin as a cosmetic agent and as a chemical peel; skin whitening agents include hydroquinone, a commonly used agent in skin whiteners. Alpha hydroxy acid (AHA) in low concentration is also used as a skin bleacher in cosmetics. In addition, kojic acid, azelaic acid, and steroids such as corticosteroids may whiten skin pigmentations. These are active agents of the composition.

### Antiacne Agents

Typical antiacne agents include retinoids, benzoyl peroxide, antibiotics, and hormonal agents. In addition, 1-Pentadecanol, as a naturally occurring long-chain alcohol, showed antiacne activity. (I. Kubo, H. Muroi, A. Kubo; J. of Natural Product. 1994 January; 57(1):9-17; “Naturally occurring antiacne agents”). It is believed that any combinations of the present invention's compounds that reduce the activity against the follicular bacterium are effective as an antiacne agent; an anti-irritant agent typically forms complexes with irritant and sensitizing compounds exemplifies soothing healing and skin protective effects, wherein the anti-irritant agent is allantoin. These are active agents of the composition.

### Flavonoids

Flavonoids occur widely in plant organs like fruits (e.g., citrus, apples, and peaches), plant extracts (e.g., milk thistle, sophora, chamomile, roman chamomile, yarrow, ginkgo, Saint John's wort licorice, etc.), leaves, flowers, and bark. They are primarily responsible for the color of the plants. The preferred flavonoid is one or more of silymarin and quercetin, or a combination thereof. The total composition having one or more flavonoids selected is from about 0.001 wt. % to about 15 wt. %, based on the total weight of the activated composition weight.

Flavonoids are polyphenolic compounds with a benzopyran skeleton as the standard chemical structure. Flavonoids are extensively used in cosmetics because of their many possible beneficial properties, such as: antioxidant, anti-free radical, antimicrobial, anti-inflammatory, enzyme inhibition, and anti-allergy; flavonoid-rich vegetable extracts are beneficial for anti-aging. These are active agents of the composition.

### Essential Oils

The composition of the present invention may contain essential oils such as Lavender oil, Peppermint oil, Chamomile oil, Lemon oil, Camphor Oil, Tea tree oil, Eucalyptus Oil, Frankincense oil, Rosemary Oil, Ginger oil, Sweet Orange oil, Lemongrass oil, Ylang-ylang oil, Lime oil, Clove oil, Bergamot oil, Rosehip oil, Thyme oil, Grapefruit oil, Myrrh oil, Geranium oil. These essential oils are from about 0.001 wt. % to about 5 wt %, based on the total composition weight. These are active agents for the composition.

### Sweeteners

The composition of the present invention may consist of the sweetening agents, including acesulfame potassium (acesulfame K), alitame, aspartame, cyclamate, fructose, glucose, isomalt, MagnaSweet®, maltitol, maltodextrin, mannitol, neohesperidine DC, neotame, Prosweet® Powder, saccharin, d-sorbitol, stevia, sucralose, sucrose, tagatose, thaumatin, xylitol, and the like. These are active agents of the composition.

### Flavors

The composition of the present invention may contain flavors, including watermelon, blueberry, strawberry, cotton candy, pineapple, peach, menthol, mango, bubble gum, apple, grape, cherry, caramel, raspberry, vanilla, coconut, lemonade, cheesecake, chocolate, tropical punch, pina colada, butterscotch, blackberry, root beer, marshmallow, orange cream, strawberry kiwi, cinnamon roll, wintergreen, butter rum, tutti-frutti, champagne, peanut butter, honey, pomegranate, vanilla butternut, mint chocolate chip, melon, English toffee, coffee, apricot, pear, licorice, butter, pecan, eggnog, maple, chocolate hazelnut, cranberry, clove, peach, craft beer, bourbon barrel, brandy, plum, pistachio, black walnut, salt water taffy, ginger flavor, or a mixture thereof. These flavoring agents are from about 0.001 wt. % to about 5 wt %, based on the total composition weight. These are active agents of the composition.

### Colorants

In one optional component of the present composition, the renewable water-insoluble surface treating composition may include various food colorants which can be used in concentrations ranging between about 0.001 wt. % and about 5 wt. %, preferably between about 0.01 wt. % and about 2 wt. %, of the total coating solution composition. Suitable colorants include yellow shade (E102), quinoline yellow (E104), orange shade (E110), carmoisine (E122), ponceau 4R (E124), pink shade (E127), red shade (E129), patent blue V(E131), indigo shade (E132), blue shade (E133), green S (E142), turquoise shade (E143), brilliant blue FCF (FD&C Blue No. 1), indigotin (FD&C Blue No. 2), fast green FCF (FD&C Green No. 3), erythrosine (FD&C Red No. 3), Allura red AC (FD&C Red No. 40), tartrazine (FD&C Yellow No. 5), sunset yellow FCF (FD&C Yellow No. 6), or a combination thereof. These are active agents of the composition.

### Nicotine and its Salts

The nicotine (CAS number: 54-11-5) compound of the present invention may include a nicotine salt and/or nicotine bases such as a nicotine source selected from the group consisting of nicotine hydrochloride, nicotine dihydrochloride, nicotine monotartrate, nicotine bitartrate, nicotine bitartrate dihydrate, nicotine sulfate, nicotine zinc chloride monohydrate, and nicotine salicylate, nicotine benzoate, nicotine polacrilex and any combination thereof. These are active agents of the composition.

### Caffeine

Caffeine (CAS number: 58-08-2) is an odorless, bitter-tasting, white crystalline purine, a methylxanthine alkaloid. Pure caffeine is a white powder that melts at 238° C. at 1 atm. Caffeine is generally less soluble in organic solvents than in hot water. Caffeine is a central nervous system (CNS) stimulant used as a cognitive enhancer, increasing alertness and attentional performance. The stimulant properties of caffeine are increased further by its enhancing effects of the natural stimulants, including dopamine, norepinephrine, glutamate, and adrenaline. The caffeine jolt usually occurs within an hour, and the effects of the caffeine will wear off in three to four hours.

Caffeine-containing beverages, for example, coffee, tea, and cola are globally in high demand. Caffeine is classified by the US Food and Drug Administration as generally recognized as safe (GRAS). These are active agents of the composition.

### Cannabinoids

Cannabinoids are compounds found in the Cannabis plant, and the best-studied cannabinoids include tetrahydrocannabinol (THC), cannabidiol (CBD), and cannabinol (CBN). (www/Wikipedia/cannabinoids) The most notable cannabinoid is tetrahydrocannabinol (THC) (CAS number: 1972-08-3), the primary psychoactive compound in the Cannabis plant. Cannabidiol (CBD) is another principal constituent of some Cannabis plants. Cannabidiol (CBD) is mildly psychotropic. Cannabinoids are purified or isolated from a Cannabis plant, particularly from its leaves to produce a substance with desired pharmacological properties. These are active agents of the composition.

### Phase Change Materials (PCMs)

The composition of the present invention may contain phase change materials (PCMs), which are reservoir materials for thermal energy as latent heat. Therefore, PCMs can absorb, store and release latent heat over a temperature range when the material changes phase. Furthermore, PCM is unique in its properties, the most important of which is the melting point.

Preferred PCMs in the present invention include natural waxes such as animal waxes such as Beeswax, Chinese wax, lanolin (wool wax), shellac wax, and spermaceti; vegetable waxes such as Bayberry wax, Candelilla wax, Carnauba wax, Castor wax, Esparto wax, Japan wax, Jojoba oil, Ouricury wax, rice bran wax, soy wax. Tallow tree wax; petroleum waxes such as paraffin wax, microcrystalline wax; or a mixture thereof. These are active agents of the composition.

### Spherical Cellulose Particles

Spherical cellulose particles (brand name of Cellets) were obtained from Transpharma Sanaq AG (Basel, Switzerland) in different sizes from about 100 μm to 1400 μm. The Cellets particles employed in the present invention are from Cellets 1000, wherein the particles are approximately 1000 to 1400 microns.

The spherical cellulose particles are prepared from microcrystalline cellulose (MCC) and provide non-porous and low surface area particles compared to the spherical activated carbon particles of the present invention. In addition, the PCM treatment, such as PCM with depressed melting point treatment via the cellulose spherical particles, is conducted in the present invention to show the process feasibility of melt coating and an aqueous dispersion of the melting point depressed Candelilla wax. The spherical cellulose particles are additives to the composition and when measured alone are comparative to the composition.

### Emulsifiers

The phase change material (PCM) of the present invention can be used as an aqueous dispersion containing one or more of an emulsifier such as Carbomer interpolymer, Carbomer copolymer, cholesterol, diethylene glycol stearate, ethylene glycol stearates, glyceryl monolinoleate, glyceryl monostearate, glyceryl distearate, lanolin alcohols, lecithin, mono- and di-glycerides, Poloxamer, polyoxyethylene 50 stearate, polyoxyl 10 oleylether, polyoxy 20 cetostearylether, polyoxyl 35 castor oil, polyoxyl 40 stearate, polyoxyl stearylether, polysorbate 20, polysorbate 40, polysorbate 60, polysorbate 80, propylene glycol monostearate, sodium cetostearylsulfate, sodium stearate, sorbitan monooleate, sorbitan monostearate, sorbitan trioleate, sodium lauryl sulfate, sorbitan monolaurate, sorbitan mono-palmitate, sorbitan sesquioleate, stearic acid, or a mixture thereof. These are active agents of the composition.

### Superabsorbent Polymer (SAP)

Superabsorbent Polymer (SAP) is a non-toxic, water and other fluid such as saline absorbing polymers such as partially crosslinked sodium polyacrylates. (See, Buchholz, F. L. and Graham, A. T., “Modern Superabsorbent Polymer Technology,” John Wiley & Sons (1998) and Lisa Brannon-Peppas and Ronald S Harland, “Absorbent Polymer Technology” Elsevier (1990)). The current commercially available granular SAP particles from gel polymerization are irregular-shaped, angular, and sharp, edgy particles from about 100 μm to 850 μm. They are prone to break through the thin back sheet films (also called pinholes) in the hygiene articles, such as diapers and other articles, when diapers are pressed to reduce their volume in packing. Diapers and other hygiene articles are exposed to pressures at wearing as well. Pinholes are recognized as the potential source of liquid leakages but more significantly, also potentially worsen the malodors issues.

More recently, the spherical-shaped SAP particles have gained market share due to their unique properties dealing with spherical geometry. For example, Aqua Keep (a tradename from Sumitomo Seika Chemicals Co Ltd, Tokyo, Japan), produced from reverse phase suspension polymerization, represents a bead SAP. It is an aggregate of agglomerated bead SAP particles with a mean diameter of about 350 μm and smaller primary bead particles from about 10 μm to 100 μm. Another round-shaped SAP particle, SAVIVA (a registered trademark of BASF AG, Ludwigshafen Germany), are individual particles from approximately 150 μm to 850 μm with less than 5% smaller than 150 μm particles and prepared by droplet polymerization. Both spherical SAP particles are characterized by non-angular and non-edgy particles in contrast to the current granular SAP particles, representing most global production. These are additives to the composition.

### Spherical Activated Composition of the Present Invention

The composition of the present invention comprises a spherical activated carbon particle having a sphericity greater than 0.6 and at least one active agent present. This active agent can be a wide variety of moieties that are added to the surface or impregnated into the spherical activated carbon particle of the present invention. Once the active agent is present, the present composition has formed. Such composition has many uses depending on the active agent selected. For example, it can be used as a highly effective composition for malodors, complex fishy smells, complex sulfur-containing malodors (represented by Kimchi malodors), including volatile acid malodors, volatile aldehydes, and VOC (volatile organic compounds), including formaldehyde.

As described earlier, when another moiety is added (compound, molecule, polymer) as an additive to the composition, it forms an admixture with the composition and not an active agent of the composition; whereas the active agent is a part of the composition by joining to the surface or entrapped or otherwise becoming a part of the spherical activated carbon particle.

The composition of the present invention has several advantages, including: sufficiently spherical geometry of the spherical activated carbon particle, which is beneficial for higher packing density in bulk and better handling; non-destructive nature to the thin films, which can play a role when included in absorbent articles; high surface areas for fast uptake of malodorous gas molecules in the air, but is not brittle in contrast to the fine powders; homogeneity of the distribution of the various active agents in the composition matrix due to the spherical geometry; possibility to load and store the active agents in the inner region of the composition to deliver and/or release in a controlled way upon being triggered in the articles during use; and a great variety of the possible active agents that can be used for various applications.

The present invention relates to a composition having a spherical, activated carbon particle, wherein the particle has sphericity greater than 0.6, preferably greater than 0.80, more preferably greater than 0.90.

Additionally, the present composition has a mean particle size d(0.5) from about 0.01 μm to 5000 μm, preferably d(0.5) from about 1 μm to 2500 μm, more preferably from d(0.5) from about 100 μm to 1500 μm, most preferably a mean particle size d(0.5) from about 300 μm to 1200 μm.

Furthermore, a composition of this invention has a particle size distribution (d(0.9)/d(0.1)) of 2.8 or less, preferably of 2.4 or less. In some of the present compositions, the particle size distribution is about 2.0 or less, including values between about 2.0 to about 1.3 or less.

In some compositions of the present invention, the composition has a particle having a BET-specific surface area of about 100 to about 2500 m²/g, preferably about 100 to 2000 m²/g, more preferably 400 to 1800 m²/g, most preferably about 800 to 1500 m²/g.

Also, the composition further comprises an iodine adsorption capacity of about 100 to about 2500 mg/g, preferably about 400 to 1800 mg/g, more preferably 400 to 1800 mg/g, most preferably about 500 to 1300 mg/g.

In the present invention, the composition further comprises a carbon tetrachloride capacity of about 30 to about 95%, preferably about 40 to about 92%, more preferably about 60 to about 87%, and most preferably about 70 to about 85%.

In one embodiment of the present invention, the composition comprises (a) about 90 wt. % to 99.9 wt. % of spherical activated carbon particles comprising sphericity of at least 0.6, a particle diameter of about 50 μm to about 2500 μm, a BET-specific surface area of about 100 to about 1500 m²/g, or further an iodine adsorption capacity of about 100 to about 2500 mg/g, and a carbon tetrachloride capacity of about 30 to about 90%, and (b) about 0.01 wt. % to about 10 wt. % of water, all based on the weight of the composition.

### Spherical Activated Composition Comprised of Activated Carbon Powder

In one embodiment of the present invention, the composition may be prepared using a powder ingredient made of activated carbon powder, activated carbon granule, spherical activated carbon particles, or a combination thereof, and a water-soluble binder.

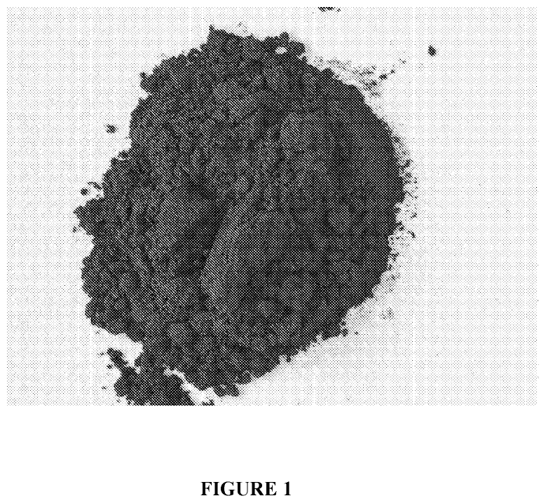

The commercially available primary raw material used for activated carbon powder is typically organic material with high carbon content, such as coconuts, nutshells, wood, peat, coal, and pitch. Activated carbon is sold in the form of powder, granule, and sphere, wherein the latter is also termed bead. (See FIG. 1)

In another embodiment of the present invention, the activated carbon powder particles useful in the present invention are not specifically limited but may be selected from the group consisting of a particulate form, a fiber form, or a mixture thereof, and comprising activated carbon, granular activated carbon, spherical activated carbon, graphene, carbon microfiber, carbon nanofiber (CNF), carbon nanotubes (CNT), and xerogels such as carbon aerogel, or a mixture thereof.

Moreover, the composition of this invention preferably comprises the activated carbon powder particles in an amount of from 0.01 to 99 wt. %, more preferably from 10 to 95 wt. %, more preferably from 25 to 90 wt. %, and most preferably from 50 to 85 wt. %, all based on the weight of the composition. When the content of the activated carbon powder particles is too low, the composition would have insufficient volatile organic compounds (VOCs). On the other hand, when the content of the activated carbon powder particles is increased, the composition may shed some of the activated carbon powder particles during the various processing steps, may cause dust, have processing issues, and permit undesired black powder particles in the end products.

In one embodiment of the present invention, the water-soluble polymer comprises one or more of a carboxylate and/or aldehyde and/or hydroxyl group. This water-soluble polymer particle contains one or more of a carboxylate and/or aldehyde and/or hydroxyl group having a molecular weight of 2000 to 2,000,000 g/mol, preferably 6000 to 800,000 g/mol, and more preferably 20,000 to 400,000 g/mol. The composition of this invention preferably comprises the water-soluble polymer particle in an amount of from 0.01 to 25 wt. %, more preferably from 0.1 to 15 wt. %, more preferably from 0.5 to 10 wt. %, and most preferably from 1 to 8 wt. %, all based on the dry weight of the composition.

In another embodiment of the present invention, wherein the composition comprising activated carbon powder and a water-soluble binder is further subjected to granulation technologies such as agglomeration, wet extrusion, and subsequent spheronization; wherein the powder and liquid form ingredients are blended adequately before granulation or extrusion, followed by drying and sizing.

### Activated Composition Comprised of Polymer Resin-Based Activated Carbon Particle

In one embodiment of the present invention, the composition comprises a spherical porous carbon particle obtained from the carbonization and subsequent activation of phenolic beads made from phenolic formaldehyde resins, ion exchange beads, porous polymer beads, hydrophobic porous polymer beads such as Dowex® Optipore L323, Dowex® Optipore V503, and Dowex® Optipore V439 brand resins (The Dow Chemical Company), and a combination thereof, by subjecting them to heat treatment in an inert atmosphere at a temperature range of 500 to 1000° C., and followed by further activation treatment, wherein the spherical porous carbon particle does not contain any binders. An example of activated carbon particles derived from polymer-based beads, such as ion exchange beads, is PureCarbon L, which is produced by PureSphere Co., LTD., South Korea.

Spherical Activated Composition Comprised of Carbon Derived from Pitch

In the most preferred embodiment of the present invention, the composition comprises a highly spherical porous activated carbon particle made from pitch material with no binders.

The spherical activated carbon particle of the present invention is further characterized by high purity, which prevents contamination of other products; low metal impurities, which come from the raw petroleum pitch materials; high-fill capability due to the increased bulk density when compared to coal-based activated carbon or coconut shell based activated carbon in the same volume capacity; high flowability due to the spherical shape; extremely low carbon dust due to the high strength and high wear resistance which prevents carbon contamination to other products; and narrow particle size distribution, which provides consistent quality for formulation. A source of such particles is Kureha Corporation, Brochure—Bead-shaped Activated Carbon (BAC) from Pitch.

### A Plurality of Active Agents

In one preferred embodiment of the present invention, the composition comprises a plurality of each active agent independently for effective malodors and volatile organic compounds (VOCs) control, including formaldehyde.

In one preferred embodiment of the present invention, the composition comprises a plurality of each active agent independently, including: water; an acid; a base; urea; a colloidal particle; an auxiliary odor control agent; an active release agent selected from the group of a bioactive agent, surface active agent, medicinally and/or hygienically active agent, cosmetically active agents, or stimulating agents such as nicotine and its salts, caffeine, and cannabinoids oil; essential oil, flavoring agent, sweetening agent, and flavonoids; or a combination thereof.

### Water Compounds

In one embodiment, the composition of the present invention comprises water as an active compound, which may be used implicitly for balancing the hydrophobicity and hydrophilicity of surfaces from about 0.01 wt. % to 10 wt. % based on the total weight of the composition.

### Acid Compounds

In one embodiment, the composition of the present invention comprises an acidic compound having a pH of 6 or less, wherein the acid compound is composed of one or more acids selected from the group consisting of hydrochloric acid, hydrobromic acid, boric acid, nitric acid, sulfuric acid, phosphoric acid, mono- and di-carboxylic acids, benzoic acid, cinnamic acid, methanesulfonic acid, ethanesulfonic acid, p-toluenesulfonic acid, salicylic acid, and low molecular weight polycarboxylic acids smaller than 5000 g/mol, or a combination of such acids.

In one embodiment, the composition of the present invention comprises a carboxylic acid selected from acetic acid, citric acid, formic acid, fumaric acid, itaconic acid, maleic acid, lactic acid, methyl fumaric acid, malic acid, methyl maleic acid, oxalic acid, salicylic acid, succinic acid, 2-methyl succinic acid, phthalic acid, tartaric acid, power of apple cider vinegar, and powdered vinegar, wherein citric acid and tartaric acid are preferred because they are economical, safe, and environmentally friendly. The composition of the present invention comprises the monomeric carboxylic acid from about 0.1 wt. % to about 80 wt. % based on the total composition weight.

### Base Compounds

In one embodiment, the composition of the present invention comprises a base compound having a pH of 8 or higher, selected from the group consisting of sodium carbonate, sodium bicarbonate (Baking soda), sodium silicate (water glass), sodium hydroxide, potassium hydroxide, ammonium hydroxide, calcium hydroxide, sodium monoxide, calcium oxide, aluminum hydroxide, cesium hydroxide, magnesium hydroxide, barium hydroxide, ferric hydroxide, pyridine, and quaternary ammonium hydroxide, and a combination thereof. The preferred water-soluble pH base compound comprises sodium hydroxide, sodium silicate, and sodium bicarbonate. Sodium silicate is the preferred water-soluble pH base compound. The composition of the present invention comprises the base from about 0.1 wt. % to about 80 wt. % based on the total composition weight.

### Sodium Silicate

In one preferred embodiment of the present invention, the composition comprises sodium silicate. Aqueous sodium silicate solution is easy to handle and can be readily applied to the spherical activated carbon particle. When dried, the aqueous sodium silicate solution is converted to solid material by water loss.

In one embodiment, the composition comprises sodium silicate compound from about 0.1 wt. % to 80 wt. % based on the total composition weight.

In one embodiment of the present invention, sodium silicate solution is used as a liquid aqueous solution wherein the viscosity ranges from about 1 to 20 P (poises) to mix with the activated composition ingredients.

In one embodiment of the present invention, the sodium silicate solution is modified by adding sugar, glycerin, and propylene glycol to promote the retention of moisture in the sodium silicate treating layer and increases its flexibility and toughness, which may help reduce attrition and dust during the processing of the composition.

In one embodiment of the present invention, sodium silicate solution is cured (solidified) by heating that can be performed by direct and indirect heating before, during, or after the addition of the silicate solution to the activated composition substrate, wherein the heating temperature is preferably gradually increased from a lower temperature to between 30 to 180° C. to evaporate water and thus to set the condensation polymerization.

In one embodiment of the present invention, sodium silicate water solution may be used to bind and agglomerate smaller spherical activated carbon particles to make larger agglomerated granules (raspberry-type granules), wherein the larger granules are robust and show little attrition.

In one embodiment of the present invention, sodium silicate water solution is used to bind and agglomerate smaller spherical activated carbon particles together with other functional particulates materials such as inorganic high surface materials such as aerogels to make larger agglomerated composite granules, wherein the larger composite granules are robust, show little attrition, and multifunctional.

### Malodor Control

A malodor or malodorous smell is an unpleasant and/or offensive odor, which may be perceived differently and can be affected by individuals, sex, culture, age group, and the environment.

It is well known in disposable absorbent articles that activated carbon and other powder types of malodor control materials are used with a binder to adhere to the surfaces of superabsorbent polymers (SAPs) or those of cellulose fluff fibers or to form a granule containing the odor control material.

As modern disposable personal care and hygiene products improve absorbency performances and leakage issues, while the thickness is getting thinner, the control of odors has become more critical to the absorbent design and consumers, including wearers, caregivers, and patients, in the same measures. Modern thinner diapers, for example, do utilize less cellulose fluff fibers. Consequently, fewer surfaces are provided in the absorbent cores with the ramifications of reduced malodor ab- and adsorption. The latter especially concerns users of feminine napkins, thin panty-liners, light incontinence products, and adult incontinence products, among others.

It is also imperative to have effective malodors and sulfur-containing VOCs control as the older population is increasing significantly. In addition, effective malodor control means will help protect the caregivers working at the elder's facilities who are unwillingly exposed to the malodorous environment.

The real difficulties of malodors and sulfur-containing VOCs control lie in the complexity and versatility of their compounds, which will be disclosed separately below.

### Chemical Natures of the Odorous Compounds

The fundamental chemical natures of the odorous compounds can be divided into acidic, basic, and sulfur-containing malodors and volatile organic compounds (VOCs), which include formaldehyde and other non-odorous compounds.

The typical acidic malodors are, for example, acetic acid, lactic acid, fatty acids such as butyric acid, n-propionic acid, n-butyric acid, n-valeric acid, isovaleric acid, and n-caproic acids; basic malodors are such as ammonia and amines such as butyl amine, trimethylamine; the sulfur-containing malodors are such as hydrogen sulfide, allyl methyl sulfide, allyl mercaptan, methyl mercaptan, dipropyl sulfide, methyl allylsulfide, dimethyl sulfide, dimethyl disulfide, dimethyl trisulfide, indole, skatole, 2-penethylfuran, thiazoles, and thiols (2-ethyl-1-hexanethiol), and volatile organic compounds (VOCs) such as ethylene, hexane, methyl pyrrole, pyridine, bacterial VOCs and fungal VOCs.

In one embodiment of the present invention, the composition sequestrates the malodors and VOC gas molecules, representing many of the typical malodors dealing with people's daily lives, such as menstruations, vaginal discharge; feces, urine; sweat odor; body odor; pet odors; trash and waste; tobacco smoke; kitchen odors such as the odors of garlic, onions, fish and greasy cooking odors; bathroom mold & mildew odors; fermented foods such as Kimchi, Sauerkrauts, cheeses, and wines; industrial waste treatment facilities, sewage treatment facilities, and landfill facilities; fertilizer facilities, industrial compost stations; beer fermentation factories; and agricultural livestock industries such as cow, pig, poultry, sheep, and goat; among others.

Based on the significance and possible economic effects on the consumer products, one of the most critical areas for improvements in the art are feminine napkins- and light incontinence-related malodors such as complex fishy smells, malodorous sulfur-containing compounds in numerous applications, and VOCs, including formaldehyde

Complex Malodors of Complex Fishy Smell. Menstrual Blood, and Vaginal Discharge

It is highly desirable to have a malodorous model system that resembles the complex menstrual blood and vaginal discharge with the characteristic malodors and malodor compounds. Creating such a system by mixing individual malodor compounds would represent an unwise undertaking due to the enormous versatility in the menstrual blood and vaginal discharge compositions and concentrations. A more sophisticated natural method that can reproduce the essential characteristic malodors such as fishy smell and various malodors from aldehydes would be beneficial.

### Complex Fishy Smell

In one embodiment of the present invention, the composition comprises as the active agent an effective malodor control agent for complex fishy smells in personal hygiene applications for absorbent articles.

The actual cause of the fishy smell of women's menstruation is complex and cannot be simply attributed to simple amines from food digestion. Furthermore, it was challenging to test and evaluate the performances of new materials due to the lack of a reliable model system for complex fishy smells. Therefore, it is highly desirable to substitute the simple amine type compound with a more sophisticated compound that may better represent the actual complex characteristics of fishy smell in real-life cases like feminine napkins, panty liners, tampons, and light incontinence applications.

In the present invention, surprisingly, it was found that commercially available marine omega-3 oil, also called fish oil or omega-3 fatty acid, suffices these requirements. Moreover, it is further found that the starting marine omega-3 oil as a natural compound produces complex malodors when exposed to the air phase at room temperature, giving a fishy smell resembling the complex fishy smell for women's menstruation-related and vulva malodors. Therefore, the commercially available marine omega-3 oil is a reliable source for complex fishy smell malodors and thus provides a unique and effective testing method for the odor control performance for feminine napkins, tampons, panty liners, light incontinence diapers, adult incontinence diapers, and wound dressings, among others.

Marine omega-3 oil is derived from fish oil and contains two marine omega-3 oils: docosahexaenoic acid (DHA) and eicosapentaenoic acid (EPA). DHA and EPA dietary sources are fatty fish, such as salmon, sardines, herrings, mackerel, and trout, and shellfish, such as mussels, oysters, krill, and crabs. Marine omega-3 oils differ from plant sources of omega-3 fatty acids, such as flaxseed, as they contain long-chain polyunsaturated fatty acids (PUFAs), EPA, and DHA. Notably, these two polyunsaturated fatty acids (PUFAs), EPA and DHA, do not have amine functional groups. However, it is a general experience that marine omega-3 oil is associated with fishy smelly belching (burping) when ingested.

Polyunsaturated fatty acids (PUFAs) are chemically unstable due to their large number of double bonds and their position within the fatty acid chain. These oils are highly prone to oxidation to lipid peroxides, which are unstable and further degrade to secondary oxidation products like aldehydes such as 4-hydroxyhexenal and malondialdehyde. (H. Esterbauer, “Cytotoxicity, and genotoxicity of lipid-oxidation products”, Amer. J. of Clinical Nutrition, 1993:57(5):779-785). The rate of lipid peroxidation is influenced by light, heat, and oxygen concentration, even under standard room conditions. As a result, marine omega-3 oil is a complex mix of EPA, DHA, other fatty acids, additives, and an unspecified concentration of lipid peroxides and aldehyde as secondary oxidation products. (Albert B Benjamin et al., “Oxidation of Marine Omega-3 Supplements and Human Health”, BioMed Res. International, Volume 2013, Article ID 464921)

Some commercial marine omega-3 oils may have inherent malodors, including a fishy smell, which can be attributed to contamination from metabolites, the spoilage of fish protein, or oxidation products of the oil itself. (M. E. Stansby, “Flavors and odors of fish oils”; J. of the Amer. Oil Chemists Soc. volume 48, pages 820-823 (1971)).

It is also noteworthy that marine omega-3 oils can be chemically oxidized, for example, via enzymatically lipoxygenase or a copper (II) salt, respectively, to form compounds that give foods a fishy smell. When EPA and DHA were oxidized with oxygen, 11 compounds in different ratios were found, from which most compounds were unsaturated aldehydes, including epoxydecadienal, formed via hydroperoxides. (Chemical & Engineering News, Volume 91, Issue 37, Sep. 16, 2013). Notably, each oxidized compound did not smell fishy per se. Still, these marine omega-3 oils can be degraded via enzymatic or chemical oxidation to form compounds that give a fishy smell in a mix and the right concentration. The fishiest odor was produced by EPA oxidized by copper (II) salt.

It is also surprising that simple oxidation of marine omega-3 oil can effectively produce a variety of complex aldehydes, which resembles the complex fishy smell of biological fluids (such as menstrual blood and vaginal discharges). It is further believed that the aldehydes will partly oxidize to fatty acids as time passes. Thus, marine omega-3 oil used as a malodor source for odor control efficacy testing can show benefits over the known fish odor simulation via amines such as trimethyl amine (TMA) and ammonia. Furthermore, the marine omega-3 oil comprising EPA and DHA represents far more complex biological molecules than small molecules like TMA and ammonia. Therefore, the fishy smell malodors created from the oxidation of EPA and DHA are reproducible, far more complex, and remarkably close to the actual situation: The complex fishy smell used in the present invention is perceived as very pungent, though not consisting of amines or ammonia.

The commercially available marine omega-3 oils supplements taken for health benefits are protected from oxidation by gelatin gel capsules and the internal use of antioxidants. Therefore, they should be sufficiently free from the inherent oxidative malodors. Indeed, when the supplement marine omega-3 gelatin capsule was opened freshly, there was merely a negligible fishy smell. In contrast, with time, the intensity of the complex fishy smell increased, indicating an oxidation reaction of polyunsaturated fatty acids (PUFAs), EPA, and DHA, at room temperature. Thus, commercially available marine omega-3 oil represents a valuable and effective but, at the same time, highly complex and sophisticated, no amine containing, fishy smell malodor source that are otherwise almost impossible to attain.

In one embodiment of the present invention, the complex fishy smell for feminine menstrual blood, vulva areas, and vaginal discharges comprise the malodors derived from marine omega-3 malodors, containing no amines and/or ammonia. This method was what was used for testing this composition.

### Volatile Sulfur-Containing Malodors and VOCs in Diverse Areas

In one embodiment of the present invention, the composition comprises an effective malodor control agent for sulfur-containing malodors and VOCs. As described above, sulfur-containing malodors and VOCs play a significant role in the odor control field due to their frequent occurrences in numerous areas and difficulties to control due to their chemical nature. Sulfur-containing malodors and VOCs may happen from both food digestion and bacterial metabolisms. The latter is also why sulfur-containing malodors and VOCs occur extremely frequently in hygiene applications such as feminine napkins, adult incontinence diapers, light incontinence diapers, health care areas such as wound dressing and oral hygiene, agricultural, and industrial applications, among others.

However, despite the tremendous efforts shown in the art, an effective method to control malodorous sulfur-containing compounds is lacking. That is also why sulfur-containing malodors from a wide range of consumer product applications such as feminine hygiene, bathrooms, oral care, and wound care are often masked via fragrances and perfumes. For example, air fresheners, perfumes in feminine hygiene products, or any other absorbent articles have been very popular in the recent past globally, where fragrances are delivered. In the case of air fresheners, the delivery means have evolved from spraying to more timely controlled release via encapsulations and controlled evaporation via the selection of proper carriers such as gels, solvents, waxes, and porous adsorbents, among others. However, a ramification is that fragrances and other cosolvents may substantially increase the VOCs when used in closed spaces with little air circulation. In the case of personal hygiene articles, the higher awareness of the possible VOC concerns has reduced the further utilization of fragrances in those articles to some degree.

As seen above, despite the significant efforts made in the recent past, the existing malodor control methods are incapable of sufficiently reducing sulfur-containing malodor and VOCs and have other drawbacks. Therefore, it is highly desirable to have a composition with effective sulfur-containing malodors and VOC control capability without using fragrances and perfumes.

Many previous approaches used a sulfur-containing compound, dimethyldisulfide (DMDS), to simulate complex sulfur-containing malodors for a given application. However, the sulfur-containing malodors can be very complex and a mix of various sulfur-containing malodor compounds. Therefore, a sulfur-containing compound can be a weak representation of the complex malodor system.

In one preferred embodiment, the composition of the present invention comprises, as an active agent, an effective malodor control agent for volatile sulfur-containing compounds, which comprises those derived from Kimchi, wherein the volatile sulfur-containing compounds include allyl mercaptan, methyl allyl sulfide, dimethyl disulfide, diallyl sulfide, methyl trisulfide, and diallyl disulfide, a combination thereof.

### Wine Malodors

In one embodiment of the present invention, the composition sequestrates the malodors and malodorous VOC molecules from wine.

In one embodiment of the present invention, the composition comprises a cork for wine bottles, wherein the composition is encapsulated in the cork, which can be natural cork and/or plastic material; wherein the composition is placed inside any type of container; further, wherein the composition of the present invention is enclosed in a polymer film sachet protected from getting wet and attached to the bottle of the cork inside the bottle, in such a way that the off-odors of wine can be adsorbed over time during storage.

### Mouth Malodors

Sulfur-containing malodors and VOCs also play a role in other personal areas, such as the mouth. For example, Halitosis is a crippling social problem, and standard dental cleaning and mouthwashes often provide only temporary relief. In addition, perfumes, colorants, and sugar contained in the mouthwash solution often offer additional challenges in dealing with tooth coloring and caries.

In one embodiment, the composition of the present invention comprises, as an active agent, an effective malodor control agent for volatile sulfur-containing compounds derived from mouth malodors. A method is presented in which the oral malodors stemming from sulfur-containing malodors producing bacteria can be counteracted by the composition of the present invention containing essential oils, zinc chloride, and cetylpyridinium chloride, wherein the composition is enclosed in a confined container such as a sachet and/or pouch made of a flexible material such as a non-woven textile and/or soft and flexible semi-permeable membranes and/or films.

In one embodiment of the present invention, the composition comprises a method of treating oral tissue inflammation. A method of treating oral tissue inflammation comprises one or more oral active agent ingredients selected from the group consisting of an anti-tartar agent, a whitening agent, a desensitizing agent, a vitamin, a compatible enzyme, a breath freshening agent, and a malodor preventing agent, and combinations thereof. Such method can use one or more components selected from the group consisting of viscosity modifiers, diluents, surface active agents, pH buffers, flavonoids, sweeteners, food flavors, colorants, preservatives, and combinations thereof.

In one embodiment of the present invention, oral tissue inflammation is associated with a condition selected from the group consisting of tobacco-induced diseases such as smoking cigarettes, cigars, and smokeless tobacco such as nicotine pouches and snus, tooth loss, oral surgery, endodontic pathoses, stomatitis, alveolar bone resorption, lesions, gingivitis, periodontitis, and combinations thereof. A method that use the present composition, as an active agent, a mouth pouch or sachet, wherein pouch or sachet is composed of cellulose-based non-wovens, chewing gum, a soluble film, a medicament gel, toothpaste, a tooth powder, or combinations thereof.

### Wound Dressing and Malodors

In one embodiment of the present invention, the composition comprises a wound dressing with an effective wound malodor control capability. The composition counter acts overpopulated malodors producing bacteria and comprise essential oils, zinc chloride, and cetylpyridinium chloride, wherein the composition is enclosed in a confined container such as a sachet and/or pouch made of a flexible material such as a non-woven textile and/or soft and flexible semi-permeable membranes and/or films.

### Ostomy/Colostomy Bag and Malodors

In one embodiment of the present invention, the composition comprises an ostomy and colostomy bag with an effective malodors control capability. The composition of the present invention is used in a confined container such as a sachet and/or pouch made of a flexible material such as a water-proof film or attached to a non-woven that can be attached to the colostomy bag surface via adhesives.

### Antiflatulence for Volatile Sulfur-Containing Compounds

In one embodiment, the composition of the present invention comprises an effective malodor control agent for volatile sulfur-containing compounds derived from flatulence malodors and wounds treatment. The composition of the present invention is used in an absorbent pad designed for absorbing gases from flatulence.

### Kimchi Smell as a Complex Pool of Volatile Sulfur-Containing Malodors

The smell of Kimchi, caused by volatile organic compounds (VOCs) with functional groups such as sulfur, aldehyde, and acids, represents a complex malodor system that cannot be reproduced easily in the lab by mixing some known sulfur compounds. Thus, it is a highly desirable functional material with deodorizing effects for Kimchi malodors.

In one embodiment of the present invention, volatile compounds identified in Kimchi, including allyl mercaptan, methyl allyl sulfide, dimethyl disulfide, diallyl sulfide, methyl trisulfide, and diallyl disulfide, are employed as a complex pool of volatile sulfur-containing malodors. The composition of the present invention comprises an effective malodor control agent, as the active agent, for volatile sulfur-containing compounds derived from Kimchi.

Bad Odors from Washable (Reusable) Menstrual Underwear

Despite the antibacterial treated fabrics, the eco-friendly period panties develop malodors due to the limitations of the washing temperatures and the wearing off of the antibacterial efficacy over the wash cycles. The composition comprises washable period underwear prepared as an insertable containment, which can be inserted into the open pouch or placed inside the underwear.

In one embodiment, the insertable containment comprises a thin layer consisting of a flexible non-wovens sachet, a thin, flexible sandwich layer consisting of porous and breathable polyvinyl alcohol films, a thin absorbent core sheet, or a thin washable and reusable textile fabric cloth.

In another embodiment, the insertable containment comprises a thin layer of an open-cell structured foam consisting of polyurethane, polyolefin, or cellulose-based elastic foams.

The insertable containment can be reused, washed, and/or disposed of, wherein the activated composition particles can be fixated in the containment via eco-friendly adhesive and/or sewing.

In one embodiment, the insertable containment comprises the composition from about 0.0005 g/cm² to 0.8 g/cm².

Bad Odors from Footwear

In one embodiment of the present invention, the composition is used to sequester bad foot odors, wherein the foot malodors are primarily acidic VOCs. The composition of the present invention is effective when applied to malodor control of a shoe and provides a shoe that deodorizes food malodors.

The deodorized shoe comprises a pouch containing the composition. The disposable pouch comprising air permeable nonwoven can be placed inside the shoe cavity or attached to the shoe inside via an adhesive layer.

Alternatively, the composition is integrated into the shoe insole through the thin coating and/or embedded during the mold foaming process.

In one embodiment, the insertable containment comprises the composition from about 0.0005 g/cm² to 2 g/cm².

### Formaldehyde, VOCs, and Volatile Aldehydes

One embodiment of the present invention comprises an effective VOC (volatile organic compound) control agent for formaldehyde.

Recently, consumer disposable absorbent hygiene products such as feminine hygiene products were found to contain various chemicals, volatile organic compounds (VOCs), including aldehydes, alkanes, aromatics, halohydrocarbons, terpenes, ketones, esters, and others. (Volatile organic compounds in feminine hygiene products sold in the US market: A survey of products and health risks; N. Lina et al., Environment International 144 (2020) 105740).

Long-term exposure to these compounds may induce skin and mucous membrane irritation, wherein highly permeable and sensitive vaginal and vulvar tissues show high uptake rates and sensitivity to chemicals and irritants. (The current guidance level of formaldehyde in absorbent hygiene products by the European Disposables and Nonwovens Association (EDANA) is 16 mg per kg (the dermal penetration based on dermal absorption estimate and toxicity clarification). (EDANA Dossier: Absorbent Hygiene Products (AHPs) A compilation of AHPs key facts with a focus on the Analysis and Risk Assessment of CODEX TM listed trace chemicals, April 2022). Therefore, despite the incomplete analysis of the possible origins, the level of formaldehyde and VOCs in hygiene products must be extremely low or absent to protect users.

In one embodiment of the present invention, the formaldehyde problem is solved by improving formaldehyde scavenging. The composition of the present invention comprises, as an active agent, urea, and a hygroscopic agent. The composition is highly effective in reducing formaldehyde.

Another embodiment of the present invention is to provide a highly effective, easy-to-handle, and highly safe formaldehyde scavenger that efficiently scavenge formaldehyde released from the home and building materials described above or the like or from the absorbent hygiene articles.

In one embodiment of the present invention, the composition comprises, as an active agent, a formaldehyde scavenging compound, including urea or a urea derivative, including biuret, triuret, cyanuric acid, and cyanuric chloride; glyoxylic acid and glycolaldehyde; amino acids such as glutamate, glycine, alanine, and sarcosine; β-diketones such as malonic acid; or a combination thereof, wherein the preferred formaldehyde scavenging compound comprises urea.

The reactions of the urea and formaldehyde result in mono methylolurea, which are believed to give both branched and linear polymers and the three-dimensional matrix due to the functionality of the urea and the formaldehyde.

The urea compound is used from about 0.1 wt. % to 80 wt. % based on the total composition weight.

In one embodiment of the present invention, the composition comprises further a hygroscopic agent and an antibacterial agent as active agents; wherein the hygroscopic agent is one or more of glycerin, propylene glycol, food-grade sugar alcohols such as sorbitol, mannitol, isomalt, xylitol, and erythritol, or a combination thereof; wherein the antibacterial agent is one or more of zinc (2+) ion and copper (1+) ion, an ionic silver in various forms: a metallic colloidal silver, or chlorhexidine: or a mixture thereof.

The hygroscopic agent is used from about 0.1 wt. % to 50 wt. % based on the total composition weight, wherein the hygroscopic agent keeps the urea more effective and from drying out.

The antibacterial agent is used from about 0.001 wt. % to 5 wt. % based on the total composition weight, wherein the antibacterial agent inhibits the decomposition of urea by bacterial decomposition into carbon dioxide and ammonia.

### Volatile Organic Compound (VOC) Control Agent

One embodiment of the present invention comprises an effective VOC (volatile organic compound) control agent for ethylene, acetylene, n-hexane, n-heptane, n-nonane, n-decane, n-undecane, n-tridecane, n-tetradecane, 3-methyl hexane, 2-methyl hexane, and methylcyclohexane, n-heptane, terpenes such as α-pinene and limonene, acrolein, methacrolein, methyl vinyl ketone, eucalyptol, linalool, citral, camphor, aromatic hydrocarbons, e.g., benzene, toluene, styrene, cresol, xylene, naphthalene, ethylene glycol, methylene chloride, tetrachloroethylene, or 1,3-butadiene, or a combination thereof.

In another embodiment of the present invention, the composition may also be used for fruit and flower packaging applications. In addition, the excellent VOC adsorption capability of the composition particles may keep the concentration of ethylene gas low. Ethylene is a gas that fruit and flowers produce and is known to be responsible for premature fruit and flower ripening. Therefore, keeping fruits and cut-flowers fresh longer may have implications of positive economic and environmental effects.

In another embodiment of the present invention, the composition for fruit and flower packaging contains a metal comprising Ag, Pt, Cu, and potassium permanganate, which is expected to provide good catalytic materials for ethylene oxidation at room temperature, these catalysts convert ethylene to carbon dioxide.

### Cigarette Smoke's Volatile Organic Compound (VOC) Control Agent

It is known that the increased risk of cancer and respiratory disease from exposure to cigarette smoke is attributed to cigarette smoke's volatile organic compounds (VOCs). For example, polycyclic aromatic hydrocarbons (PAHs) and volatile N-nitrosamines represent the major carcinogens in tobacco smoke, while additional toxic chemical compounds such as carbon monoxide, benzene, arsenic, and formaldehyde further harm the health of smokers and secondary smokers significantly.

In one embodiment of the present invention, the composition relates to smoke cigarette filters. The present invention may offer a method that reduces cigarette smoke's hazardous VOCs, including formaldehyde.

One advantageous embodiment of this invention is that the composition comprises particles with a spherical shape and a diameter from 100 μm to 2000 μm.

### Auxiliary Odor Control Agent

In a preferable embodiment, the composition of the present invention comprises an auxiliary odor control agent that is added as a powder as an additive and the soluble metal salts in the form of an aqueous solution as an active compound to the spherical activated carbon particle.

The powdery odor control agent is preferably used from 0.01 to 80 wt. % based on the total weight of the dry composition, wherein the powdery odor control particles have a particle diameter of 10 μm or more and less than 500 μm.

The soluble metal salt-type auxiliary odor control agent such as metallic or ionic silver in various forms such as colloidal silver nano-particles, silver acetate, silver nitrate, or silver thiosulfate complexes are preferably used in an amount from 0.001 to 15 wt. %, more preferably from 0.025 to 10 wt. %, and most preferably from 0.01 to 5 wt. %, based on the total weight of the dry composition.

In another embodiment of the present invention, mixtures of powdery odor control agents of different types can be employed. The powdery odor control agent is added as a powder simultaneously, blending the spherical activated carbon particle and other powder form additives before or after. For example, the metal salt auxiliary odor control agent is typically added as an aqueous solution by wetting the spherical activated carbon particle via known processes, such as mixing, blending, or fluidized bed.

In another embodiment of the present invention, the drying includes heating at a temperature from about 50° C. to 300° C. for 1 minute to 360 minutes utilizing an air convection oven dryer, belt dryer, drum dryer, fluidized bed dryer, microwave dryer, UV dryer, or a combination thereof.

### Colloidal Particles

In one embodiment of the present invention, the malodor control composition comprises colloidal particles, wherein preferred particles are fumed silica of hydrophobic or hydrophilic properties or superhydrophobic silica aerogels.

In one embodiment of the present invention, the colloidal particle is used in the concentration of 0.001 wt. % to 15 wt. %, based on the weight of the water-insoluble surface treating agent solution.

In one embodiment of the present invention, the colloidal particle comprises particles having primary particle diameters of 1 nm or more and less than 10 μm, wherein the colloidal particles have a particle diameter of 0.001 μm or more and less than 100 μm in the content of 0.001 to 10 wt. %, based on the total weight of the absorbent core.

In one embodiment of the present invention, the colloidal particle comprises high surface porous particles with a BET-specific surface area of 10 to 2500 m²/g and a 10/c or more porosity.

### Active Release Agents

In one embodiment of the present invention, the malodor control composition comprises an active agent released during use, wherein such release can be controlled and triggered by liquid contact and/or temperature change or a combination thereof.

In one preferred embodiment of the present invention, the active release agent comprises a bioactive agent, a surface-active agent; a medicinally and/or oral hygienically active agent; a cosmetically active agent; a stimulating agent such as nicotine and its salts, caffeine, a cannabinoid; a flavonoid, an essential oil: a flavor; or a mixture thereof.

In the present invention, the bioactive agent comprises one or more of an antibacterial metal ion such as zinc (2+) ion, copper (1+) ion, silver (+1) ion, and a metallic colloidal silver; an anti-viral agent, an antimicrobial agent; or a mixture thereof, and is used in the concentration of about 0.01 wt. % to about 80 wt. %, based on the total activated composition weight.

Other release agents of the present composition are: Flavonoids; Essential Oils; Flavors; Sweeteners; and Colorants. These agents are further discussed in this application.

These release agents of sweetening agents, flavors, and essential oils, can be present in the present composition from about 0.01 wt. % to about 50 wt. % based on the weight of the activated composition.

### Surface-Active Agent

In one embodiment of the present invention, the active release agent comprises the surface-active agent being an emulsifying agent comprising a lecithin, polysorbate-80, lauric acid, myristic acid, palmitic acid, stearic acid, linolenic acid, linoleic acid, oleic acid, and a mixture thereof and is used in about 0.01 wt. % to 5 wt. %, based on the total composition weight.

Medicinally and/or Oral Hygienically Active Agent

In one embodiment of the present invention, the composition comprises the medicinally and/or oral hygienically active agent used for the condition selected from the group consisting of tooth loss, oral surgery, gingivitis, periodontitis, tobacco- and nicotine-related mouth malodors and inflammations, nicotine-pouch-use-related mouth ulcers, and combinations thereof.

The medicinally and/or oral hygienically active agent of the present invention comprises an antioxidant; an anti-inflammatory agent such as an extract of magnolia or clove; an enzyme; a probiotic, or a prebiotic; a dental agent such as a whitening agent, an anti-tartar agent, a desensitizing agent; a breath freshening agent such as a flavonoid; an antimicrobial agent as a mouth malodor preventing agent such as chlorhexidine; or a mixture thereof, used in an amount of about 0.1 to 80 wt. %, based on the total composition weight.

In one embodiment, the medicinally and/or oral hygienically active agent comprises one or more components selected from the group consisting of water-soluble polymers; surface active agent agents; pH modifying agents, mouth feel agents such as spherical cellulose particles or porous hydrophobic polymer particles of specific sizes, for example, about 400 μm to 1500 μm; flavonoids; essential oils; sweetening agents; flavor agents; colorants; preservatives; and combinations thereof, used in an amount from about 0.01 wt. % to 80 wt. %. based on the total weight of the activated composition weight.

### Cosmetic Ingredients

In one embodiment of the present invention, the composition comprises an anti-wrinkle agent, including hyaluronic acid and botulinum toxin.

In one embodiment of the present invention, the composition comprises an anti-wrinkle agent used in the concentration of 0.01 wt. % to 15 wt. %, based on the total composition weight.

In another embodiment of the present invention, the composition comprises one or more of the selected from the group of alpha hydroxy acid, a skin whitening agent, an anti-acne agent, and an anti-irritant agent.

In another embodiment of the present invention, the total composition comprising one or more of selected from the group of an alpha hydroxy acid, a skin whitening agent, an antiacne agent, and an anti-irritant agent is from about 0.01 wt. % to 15 wt. %, based on the total weight of the composition weight.

### Stimulating Agent Activated Composition

In one embodiment of the present invention, the stimulating active agent comprises nicotine and its salts, caffeine, and cannabinoids, contained in an oral pouch designed for administration in the oral cavity enclosed in a saliva-permeable non-woven and/or semi-permeable membrane material.

In one embodiment of the present invention, the composition comprising a high spherical surface of activated carbon provides a proper condition for enough nicotine, caffeine, and cannabinoid compound to be stored and controlled, and released over a more extended period than the current soluble form of a caffeine powder formulation in a tablet or liquid beverage.

### Nicotine and Nicotine Salts

Nicotine, present in a tobacco plant, is a highly addictive chemical compound, changing the brain's work and causing cravings. All tobacco products contain nicotine, including cigarettes, non-combusted cigarettes (also called heat-not-burn tobacco or heated tobacco products), cigars, smokeless tobacco (such as dip, snuff, snus, and chewing tobacco), hookah tobacco, and most e-cigarettes. Tobacco products containing nicotine pose different levels of health risks to users. Combustible products that burn tobacco, such as cigarettes, are the most harmful, delivering more than 7,000 chemicals and nicotine addiction. (www.fda.gov).

The convenience of smokeless tobacco pouch (snus) and nicotine pouch has recently gained substantial popularity in Nordic Europe, replacing traditional smokers. While snus still uses the tobacco leaves, the nicotine pouch comprises nicotine, and/or nicotine salt and is entirely tobacco leaves-free. Unfortunately, with the increase of this smokeless tobacco consumption, snus and nicotine users often suffer from bad mouth odors, inflammations, and ulcers in the soft and sensitive gums in the oral cavity, due to repeated nicotine consumption and other formulary ingredients. Therefore, it is highly desired to provide methods to reduce such issues dealing with smokeless tobacco consumption.

Typically, the nicotine and other ingredients in the formula are dissolved instantly (“burst”) from the substrate without much control into the saliva, so rushed that they are exhausted in a relatively short time. At the same time, consumers desire continuous consumption over a prolonged time Therefore, controlling and/or designing the migration of nicotine and other ingredients from the substrate is highly desired.

The same observations can be made from the caffeine and cannabinoid related-application, and therefore, controlling the migration of caffeine and cannabinoid from the substrate is highly desired.

Different means may be utilized in the present invention to achieve the desired nicotine releases above: the use of fast-releasing activated composition and delayed- and controlled releasing activated composition so that there is a constant stream of nicotine over a prolonged period.

The fast-releasing activated composition can utilize smaller sizes in a given unit weight. Therefore, a blend of different sizes may be used to optimize the initial release of nicotine.

Encapsulation with PCM can delay or control the active release agent's migration from the surfaces. Activated composition with or without PCM encapsulation can also be utilized for the optimum out of nicotine and/or active release agents.

It was notable to find that the melting temperature of the composition can be designed via the melting point depression, which can be designed based on the mouth temperature and duration of use. Therefore, a blend of compositions with varying melting points can be utilized within a temperature range to warrant a smooth continuous release of nicotine over time.

The composition of the present invention provides an effective carrier for the means of controlled delivery of nicotine, oral hygiene agents, and malodor controls.

In one embodiment, the present invention relates to nicotine containing oral pouch designed for administration of nicotine and/or its salts in the oral cavity, wherein the nicotine pouch comprises a combination of the functional composition and nicotine and/or nicotine salts composition as the active agent enclosed in a saliva-permeable pouch of a non-woven and/or semi-permeable membrane material.

The present invention may offer a method that nicotine is provided to a user by effectively releasing a higher amount of nicotine and/or its salts based on the high surface areas and functional design of the composition of the present invention.

One advantageous embodiment of this invention, the composition comprises nicotine in an amount of 0.001 to 80 wt. %, based on the weight of the composition.

One advantageous embodiment of this invention is that the composition comprises particles with a spherical shape and a diameter from 100 μm to 2000 μm.

In one preferred embodiment, the nicotine of the present invention is derived from tobacco.

In one embodiment of the present invention, the nicotine compound is one or more of nicotine monotartrate, nicotine bitartrate, or nicotine bitartrate dihydrate.

In one embodiment of the present invention, the composition comprises a water-soluble polymer and may be the carrier of the nicotine and/or nicotine salt composition.

In one embodiment of the present invention, the nicotine-containing composition comprises a method of treating oral tissue inflammation of the oral gums and mucus caused by continuous use of nicotine in the oral cavity comprises one or more components selected from the group consisting of water-soluble polymers; surface active agent agents; pH modifying agents; mouth feel agents such as spherical cellulose particles or porous hydrophobic polymer particles of specific sizes, for example, about 400 μm to 1500 μm; flavonoids; essential oils; sweetening agents; flavor agents; colorants; preservatives; and combinations thereof, used in an amount from about 0.01 wt. % to 80 wt. %, based on the total weight of the composition weight.

### Caffeine

In one embodiment of the present invention, the composition comprises a Cannabis active compound and a cannabinoid controlled-release method formulation.

One embodiment of the present invention is producing a readily consumable caffeine formulation that retains the desired stimulating properties but increases mouth hygiene and malodors. The composition with caffeine of the present invention comprises an oral pouch designed for administration of cannabinoid and/or cannabinoid oil in the oral cavity, wherein the cannabinoid pouch contains a combination of the composition and cannabinoid and/or cannabinoid oil enclosed in a saliva-permeable pouch of a non-woven and/or semi-permeable membrane material.

In one embodiment of the present invention, the caffeine-containing composition comprises a method of treating oral tissue inflammation of the oral gums and mucus caused by continuous use of nicotine in the oral cavity comprises one or more components selected from the group consisting of water-soluble polymers; surface active agent agents; pH modifying agents; mouth feel agents such as spherical cellulose particles or porous hydrophobic polymer particles of specific sizes, for example, about 400 μm to 1500 μm; flavonoids; essential oils; sweetening agents; flavor agents; colorants, preservatives; and combinations thereof, used in an amount from about 0.01 wt. % to 80 wt. %, based on the total weight of the activated composition weight.

### Cannabinoids

In one embodiment of the present invention, the composition comprises a Cannabis active compound and a cannabinoid controlled-release method formulation.

One embodiment of the present invention is producing a readily consumable formulation that retains the desired pharmacological properties but increases mouth hygiene and malodors. The composition with cannabinoid of the present invention comprises an oral pouch designed for administration of cannabinoid and/or cannabinoid oil in the oral cavity, wherein the cannabinoid pouch contains a combination of the composition and cannabinoid and/or cannabinoid oil enclosed in a saliva-permeable pouch of a non-woven and/or semi-permeable membrane material.

The composition comprising a highly spherical surface of carbon provides a proper condition for enough cannabinoid compound to be stored and controlled released over a more extended period than the current soluble form of a cannabinoid powder formulation in a tablet. The user often perceives an unpleasant aftertaste from the cannabinoid extract or oil. Such unpleasant experiences can be significantly reduced or eliminated using high surface spherical activated carbon of the composition of the present invention.

In one embodiment of the present invention, the cannabinoid-containing composition comprises a method of treating oral tissue inflammation of the oral gums and mucus caused by continuous use of nicotine in the oral cavity comprises one or more components selected from the group consisting of water-soluble polymers; surface active agent agents; pH modifying agents; flavonoids; essential oils; flavor agents; sweetening agents; colorants; preservatives; optionally, mouth feel agents such as spherical cellulose particles or porous hydrophobic polymer particles of specific sizes, for example, about 400 μm to 1500 μm and combinations thereof, used in an amount from about 0.01 wt. % to 80 wt. %, based on the total weight of the activated composition weight.

### The pH Buffering Agents

In one embodiment of the present invention, the pH modifying agent is a buffering agent, wherein the preferred pH controlling agent is selected from the group consisting of citric acid, gluconic acid, lactic acid, tartaric acid, sodium orthophosphate, sodium diphosphate, sodium polyphosphate, sodium carbonate, sodium bicarbonate, magnesium carbonate, magnesium oxide, or any combination thereof.

In another embodiment of the present invention, the total composition comprising one or more buffering agents where the pH buffer is from about 0.1 wt. % to 40 wt. %, based on the total weight of the activated composition weight.

### Phase Change Materials (PCMs)

In one embodiment of the present invention, a composition is manufactured by treating spherical activated carbon particles with a phase change material (PCM), wherein the preferred PCM comprises a natural wax such as Candelilla wax, capable of melting and solidifying at the phase transition temperature.

In one embodiment of the present invention, the composition comprises a melting point depressed PCM and a lower alkyl chain hydrocarbon, such as hydrophobic MCT oil. The two substances mix easily because of their hydrophobic nature. The depressed melting point from its original melting temperature can be controlled via the type and added quantity of hydrophobic oil.

Lowering the PCM's melting temperature induced by a lower alkyl chain hydrocarbon allows the designed applicability of PCM depending on the melt temperature, such as mouth and body temperature. According to the spirit of the present invention, it is self-explanatory that melting point depressed Candelilla wax can be replaced, for example, with other waxes and other phase change materials (PCMs), respectively.

The PCM with depressed melting temperature in the present invention comprises a PCM with a melting point depression of at least 0.5° C. or greater from the virgin PCM, wherein the lower alkyl chain hydrocarbon comprises an MCT that is medium fractionate glyceride distilled from coconut oil.

In one embodiment of the present invention, an activated composition is manufactured by treating spherical activated carbon particles via a melt coating process, wherein a wax melt is formed by melting and subsequently surface treated with spherical activated carbon particles, which acts as the thin layer of PCM material, which can change their phase as temperature increases or decreases.

In one embodiment of the present invention, the coating of the thin, neat PCM or a melting point-depressed PCM layer on the surfaces of the composition is conducted by melt spraying, mixing, or melt mixing under agitation, or a combination thereof.

In one embodiment of the present invention, the PCM material comprises 0.01 to 90 wt. %, based on the total weight of the composition.

In one embodiment of the present invention, the PCM is added to the composition of this invention by mixing in a mixer, a high shear mixer, a granulator, a blender, a fluidized bed, an extruder, or a combination thereof.

In one embodiment of the present invention, the PCM comprises a free-flowing aqueous dispersion of the PCM below its melting temperature.

In one embodiment of the present invention, the aqueous dispersion of the phase change material (PCM) comprises one or more emulsifiers, wherein the preferred emulsifiers include lecithin, polysorbate 60, polysorbate 80, Carbomer copolymer, sodium stearate, sodium lauryl sulfate, sorbitan monooleate, sorbitan monostearate, sorbitan monolaurate, sorbitan mono-palmitate, sorbitan sesquioleate, steric acid, or a mixture thereof.

Aqueous PCM dispersion comprises particles with a diameter of about 0.001 μm or more and less than 100 μm in the content of 0.001 to 50 wt %, based on the total weight of the dispersion, wherein the emulsifier concentration is at least 0.01 wt. % to 10 wt. %, based on the total weight of the dispersion.

The aqueous PCM dispersion comprises a particulate anticaking agent, including one or more selected from the group consisting of cellulose powder, magnesium stearate, calcium carbonate powder, silicon dioxide, fumed silica, or a combination thereof.

In one embodiment of the present invention, the PCM-treated activated composition particles are subject to drying at slightly elevated temperature from about room to 50° C. below PCM's melting point, employing air convection oven dryer, belt dryer, drum dryer, fluidized bed dryer, microwave dryer, infrared dryer, UV dryer, or a combination thereof.

### Spherical Cellulose Particle

In one embodiment of the present invention, the composition comprises a spherical cellulose particle, representing an additional actives-carrier particle in addition to the composition of the present invention.

The spherical cellulose particles are inadequate to sequestrate the malodors by adsorption mechanism due to the lack of a high surface, unlike the current invention's composition. However, they are a suitable carrier for releasing various active agents disclosed in the present invention. Therefore, the spherical cellulose particles may be treated with the same treatments for the composition disclosed in the present invention.

The non-porous and low surface area properties of the spherical cellulose particles as carriers for odor control, compared to the present invention's high surface, may provide the activated composition particles of the present invention with more effective designing of the controlled release methods.

In one embodiment of the present invention, the composition comprises a mixture with spherical cellulose particles from about 10 μm to 2500 μm diameter sizes and from about 0.01 wt. % to 99.99 wt. % based on the total weight of the mixture. The spherical cellulose particles are non-angular, non-sharp edgy, non-abrasive, non-attritive, and therefore compatible with the composition of the present invention.

In another embodiment, the spherical cellulose particles comprise a phase change material (PCM), wherein the PCM is used for encapsulating the cellulose particles, which helps control the release of the active agents from the surfaces of the composition.

In one embodiment of the present invention, the encapsulation is conducted by applying a melt coating or aqueous dispersion of the virgin PCM or melting point depressed PCM, wherein the PCM is used from about 0.01 wt. % to 80 wt. % based on the total weight of the composition.

### Superabsorbent Polymers (SAPs)

SAP particles in modern diapers and feminine care products have become unreplaceable. Due to the irregular shape of the commercial granular, SAP particles have many issues: they are abrasive, attritive, creating dust, and causing pin holes, potentially increasing the leakage and malodors. Any materials, part of the hygiene articles, in the presence of SAP particles must possess robustness withstanding the hostile conditions caused by the current granular SAP particle morphology. The composition of the present invention was surprisingly found to be robust and attrition resistant.

The composition of the present invention may comprise a mixture with irregular-shaped granular superabsorbent polymer (SAPs) particles prepared from aqueous gel polymerization from about 0.01 wt. % to 99.99 wt. % based on the total weight of the mixture.

In one embodiment of the present invention, the composition comprises a mixture with a spherical SAPs particle such as a bead or round-shaped SAP particle prepared from reverse phase suspension and droplet polymerization from about 0.01 wt. % to 99.99 wt. % based on the total weight of the mixture. The current commercially available spherical SAP particles are non-angular and non-sharp edgy, thus compatible with the present invention's composition.

In yet another embodiment of the present invention, the composition may be used in the presence of a granular superabsorbent polymer (SAPs), wherein the granular SAP particles are preferably below about 650 μm or less in diameter. It is believed that irregular-shaped, angular, and sharp-edgy granular SAP particles of larger sizes, such as approximately 700-850 μm, are more prone to creating pinholes under pressure. Therefore, keeping the granular SAP particle sizes below 650 μm would cause fewer harmful pinholes, despite the granular nature of the current SAP particles.

In another embodiment of the present invention, the composition is characterized by the low degree or absence of segregation in the mixture with the SAP based on similar properties of sizes and particle densities.

### Uses of the Present Composition

The composition particles of this invention can be used in any application that desires the reduction of malodors and/or malodorous sulfur-containing volatile organic compounds (VOCs), and/or VOCs, including ethylene, volatile aldehydes, formaldehyde, and/or volatile acids.

The composition of the present invention is versatile in its functionality and can be beneficial for various consumer products-related applications, agricultural applications, and industrial applications.

### Consumer Products-Related Applications

In one embodiment of the present invention, the composition of the present invention may be used with cellulose fluff pulp fibers in absorbent articles, in which the spherical activated composition particles are contained.

In one preferred embodiment, the composition of the present invention may be used in absorbent cores comprising personal care applications in feminine napkins, tampons, panty liners, washable menstruation panty (reusable menstrual underpants), and incontinence underpants) for both menstrual bloods and vaginal discharges, and incontinence products, including heavy and light incontinence, baby diapers, and liquids.

In one embodiment of the present invention, the composition may be embedded in a thin cellulose fluff absorbent sheet as an insert and/or an insertable sachet in the washable menstruation panty (reusable menstrual underpants) and incontinence underpants).

In one embodiment of the present invention, the composition may also be used in pet hygiene articles such as pet diapers, pet bed pads, and cat litter.

In one embodiment of the present invention, the composition may also be used in health- and hygiene-related applications like wound dressings and fabrics, ostomy/colostomy bags, hospital gowns, bandages, blood pads, antiviral and antibacterial surgical face masks, wound dressings, antiflatulence pads or diapers, and hospital bed pads.

In one embodiment of the present invention, the composition may also be used in medical masks and gown products due to its strong antiviral and antibacterial properties and absorption, malodor, and VOC control properties.

In one embodiment of the present invention, the composition may be used in making consumer masks with potent antiviral and antibacterial properties especially desirable for Coronavirus 2019 (Covid-19).

In one embodiment of the present invention, the composition may also be used in diverse consumer malodor product applications, such as in malodors for fridge, bathroom, pet, cooking, furniture, closet deodorizers, carpet deodorizers, footwear, perspiration, clothing, smoke eaters, sportswear, firefighter's clothes, and carpet backing.

In one embodiment of the present invention, the composition may be used to reduce mouth malodors from smoke-free tobaccos such as nicotine pouches and snus, wherein the composition is enclosed in saliva-permeable sachet.

In one embodiment of the present invention, the composition comprises a method of treating oral tissue inflammation.

In one embodiment of the present invention, oral tissue inflammation is associated with a condition selected from the group consisting of tobacco-induced diseases such as smoking cigarettes, cigars, and smokeless tobacco such as nicotine pouches and snus, and combinations thereof.

In one embodiment of the present invention, the oral care composition is in a form selected from the group consisting of a mouth pouch or sachet, wherein pouch or sachet is composed of cellulose-based non-wovens, chewing gum, a soluble film, a medicament gel, toothpaste, a tooth powder, or combinations thereof.

In one embodiment of the present invention, the composition may sequestrate the malodors and malodorous VOC molecules from wine, wherein the composition is encapsulated in the cork or enclosed in a polymer film sachet.

The present composition may also be used in diverse consumer malodor product applications, such as in malodors for food packages for Kimchi. Sauerkrauts, cheeses, red meats, fish, chickens, poultry, sausages, hams, and salamis.

The present composition may also be used in fruit and cut-flower packages for ethylene-emitting produce and cut-flowers such as bananas, apples, avocados, melons, peaches, pears, tomatoes, plums, and carnation.

In another embodiment of the present invention, the composition for fruit and cut-flower packaging contains a metal comprising Ag, Pt, and Cu from about 0.0001 wt. % to 5 wt. % or potassium permanganate from about 0.01 wt. % to 95 wt. % based on the total weight of the composition, which may be potentially good catalytic materials for ethylene oxidation at room temperature, wherein these catalysts convert ethylene to carbon dioxide.

In one embodiment of the present invention, the composition may be used in cigarette filters for removing ammonia and cigarette smoke's volatile organic compounds (VOCs), including major carcinogens in tobacco smoke such as polycyclic aromatic hydrocarbons (PAHs), volatile N-nitrosamines, other toxic chemical compounds such as carbon monoxide, benzene, arsenic, and formaldehyde.

In one embodiment of the present invention, the composition may be used in home care products such as kitchen garbage bags, body bags, diaper pails, soiled laundry, laundry bags, trash containers, and dead animal containment.

In one embodiment of the present invention, the composition may be used in air filter products for odor control in typical households, schools, offices, commercial buildings, public bathrooms, restaurants, hospitals, assisted living and nursing homes, as well as in commercial applications such as cabin air filtration/freshening applications for VOCs such as automobiles, aircraft, buses, trains, trucks, RVs, ships, and boats.

The present composition may also be used in building applications such as natural mineral or synthetic insulation wool, mortar, cement, and concrete.

In one embodiment of the present invention, the composition comprises facial masks, cream, and/or lotion products in cosmetics for anti-wrinkles, chemical peel, skin whitening agents, and antiacne agents.

For these uses an article and/or product is made suitable for use in consumer, personal care, home care, and healthcare-related products and industrial applications having an activated composition of this invention from about 0.01 wt. % to about 99.99 wt. % based on the total weight of the article, wherein the article effectively sequesters one or more of: complex fishy smells derived from marine omega-3 oil; complex sulfur-containing compounds derived from Kimchi; volatile organic aldehydes, including formaldehyde; volatile organic acids; or volatile organic compounds including ethylene.

### Agricultural Applications

The present composition may also be used in gardening applications in moisture control and triggered release of active agents of fertilizers, pesticides, herbicides, fungicides, and biocides.

The present composition may also be used in agricultural and horticultural applications such as in livestock odor containment to combat the malodors, and VOC gas molecules represent many of the typical malodors dealing with animals' feces, urine from such as cows, pigs, poultry, sheep, and goat, among others.

### INDUSTRIAL APPLICATIONS

The present renewable water-insoluble surface treating composition may also be used in industrial applications such as pharmaceuticals for drug delivery, consumer products for packaging, nutrition, textiles, farming products such as fruits, nuts, seeds, and vegetables, catalysts, food processing, waste processing, water filtrations, wildfire control, and paper and pulp manufacturing.

In one embodiment, the composition of the present invention comprises an effective malodor control agent for volatile sulfur-containing compounds derived from industrial waste treatment facilities such as waste processing, wastewater handling facilities, sewage treatment facilities, landfill facilities, fertilizer facilities, industrial compost stations, among others.

The present composition may also be used in industrial applications such as catalysts, desalination facilities, (RO) filtrations facilities, wildfire control, and paper and pulp manufacturing, among others.

In one embodiment of the present invention, the composition combats the malodors, and VOC gas molecules, representing many of the typical malodors dealing with food processing of fermented foods such as sauerkraut, kimchi, cheeses, and wines; beer or sake fermentation factories; among others.

In one embodiment of the present invention, the composition can be used in applications such as dairy products, slaughterhouses, industrial and hospital laundries, and beverage industries, among others

In one embodiment of the present invention, the composition is subjected to recycling and, more particularly, to a washing and purifying apparatus capable of regenerating the composition.

In general, adsorbed malodor molecules emitted from various applications, such as hygiene, fruit packages, kitchens, housings, buildings, etc., contain different compounds and may contain harmful substances such as various malodors and pollutants. The exhaust gas of desorbed compounds is purified and released into the atmosphere to prevent contamination of the air. In the present invention, the used composition may be gathered, optionally washed, and regenerated via an adsorption tower for activated carbon-type materials, which is a well-known device in the art that adsorbs and removes adsorbed malodors and VOCs.

This invention will be further clarified by consideration of the following examples, which are intended to be purely exemplary of the invention.

### Materials and Methods Used in the Examples

### Materials

The following materials are used in the following examples. All percentages are by weight (wt. %) unless otherwise stated.

### Spherical Activated Carbon Particle

Bead-shaped activated carbon (BAC) (GAS Number: 7440-44-0) was obtained from Kureha Corporation (Tokyo, Japan) and used as the spherical activated carbon particle of the present invention. Kureha's BAC is produced from the raw material pitch with low metal impurities and no binder. BAC generates significantly lower carbon dust due to the wear and attrition resistance, which is essential to reduce black particle contamination to other products. The high purity of the BAC also prevents contamination of other ingredients of the present composition.

Two different grades of spherical activated carbon particles from Kureha were used, and their average particle size and the weight fraction of size were as follows: A-BAC-MP (0.50=0.05 mm, 0.25 mm or less 5.5 wt. %, 0.71 mm or more S 10 wt. %) and G-BAC GR-70R (0.70 mm≤, 0.6 mm or less ≤5 wt. %).

The specific surface area of the BAC measured via the BET method ranges from 1100 to 1300 m²/g.

Carbon Tetrachloride Activity (JIS K1474-5.1.2. Standard) measures the loading of carbon tetrachloride, weight percent on carbon, at concentrations close to saturation in the air. The method measures the pore volume of the activated carbon and is primarily used as a quality assurance test for producing activated carbon.

The iodine adsorption number in mg/g of activated carbon is a measure of the amount of iodine that can be adsorbed on the surface of a given mass of carbon black. (ASTM D1510-21) The iodine adsorption number is related to the surface porosity and is characteristic of the surface area of activated carbon.

Another spherical activated carbon particle, available as product grade PureCarbon L from PureSphere Co., LTD., South Korea was evaluated. These particles are created by polymerizing spherical polystyrene-based polymer beads and subsequently carbonizing and activating them. Similar to Kureha's BAC, PureCarbon L offers excellent wear and attrition resistance, reducing carbon dust generation and the risk of contamination.

The spherical activated carbon particles from PureSphere Co., LTD., Korea have an average particle size, with a weight fraction of smaller than 0.20 mm (5 wt. %) and equal to or greater than 0.2 to 0.6 mm (95 wt. %). PureCarbon L's specific surface area, determined using the BET method, falls within the range of 1000±100 m2/g. Additionally, PureCarbon L exhibits a packing density of 0.65±0.05 g/cm3, highlighting its uniform spherical nature.

### Activated Carbon Powder

Activated carbon (CAS number: 7440-44-0) powder (FIG. 1) was obtained from Samchun (Daejung) chemical, LTD.

### Sodium Bicarbonate (Baking Soda) Powder

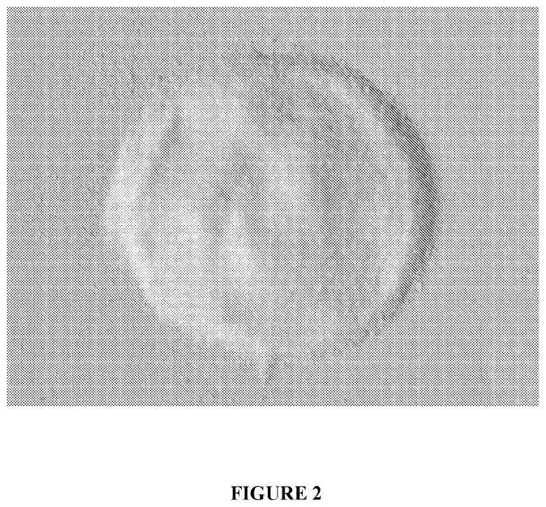

Sodium bicarbonate (baking soda; CAS number: 144-55-8) powder (FIG. 2) was obtained from Samchun (Daejung) chemical, LTD.

### Marine Omega-3 Oil (Fish Oil)

The marine omega-3 oil used in the present invention was purchased from Costco (Sejong, Korea). The supplement package contains 180 Omega-3 capsules, wherein one capsule of the weight of 1,300 mg contains 1,100 mg of both docosahexaenoic acid (DI-HA) and eicosapentaenoic acid (EPA), sourced from herrings, sardines, and mackerels. The omega-3 oil is kept at room temperature, and the capsule is cut to get the oil before testing.

### Kimchi Broth

Commercial Kimchi used was Peacock Poggi Kimchi obtained from Chosun Hotel Kimchi Company (Coupang, South Korea). The broth of Kimchi was used as the source of the Kimchi smell. The sniff testing was conducted after Kimchi was ripened with approximately two weeks' fermentation time in a fridge at about 4° C.

### Citric Acid Powder

Cosmetic grade citric acid powder (CAS #77-92-9) was obtained from SoapGoods.com, Atlanta, USA.

### Sodium Silicate Solution

Sodium silicate solution (Na2SiO3, CAS #1344-09-8) was obtained from Reagents Chemicals, Gyeonggi-do, Republic of Korea.

### Titanium Dioxide

Cosmetic grade titanium dioxide (CAS #13463-67-7) was obtained from SoapGoods.com, Atlanta, USA, and used as a fine powder after sieving with an approximately 300 μm sieve.

### Medium-Chain Triglycerides (MCTs)

The MCT oil (Chemres Technologies Inc, Quezon City, The Philippines) is a supplement made from a fractionated fat called medium-chain triglycerides and was obtained from coconut oil. Medium-chain triglycerides (MCTs) are fatty acid esters of glycerol that contain saturated 6-12 carbons. Coconut oil contains approximately 60-70% MCT. In contrast, long-chain triglycerides (LCTs) are composed of fatty acids with the length of 12-18 carbons.

### Candelilla Wax

Candelilla wax in flake form was obtained from the Korea Similac company (Coupang, South Korea). A small quantity of Candelilla wax was placed in a 50 mL aluminum container and subjected to the hot water bath (85˜90° C.). The melting wax is transparent and honey-like. The Candelilla wax showed a homogenous appearance, clear and shiny. Candelilla wax flakes' melting point was determined to be approximately 65 to 68° C. measured by a probe thermometer (PT305, Hengshui Ximei Electronics Technology Co., LTD; temp. range of −50˜350° C.). The flakes were chopped, and the fraction that passed through about 500 μm sieve was used.

### Red Colorant

The colorant used was an artificial water-soluble food coloring (McCormick's Food Coloring Red) and was used as obtained. According to McCormick's information (www.mccormick.com), the red color is a mix of water, propylene glycol, FD&C Reds 40 and 3, and propylparaben (preservative). FD&C Red No. 3 (Erythrosine) is one of nine certified FD&C (Food, Drugs & Cosmetics) color additives commonly used as a cherry-red coloring in candy, popsicle, and cake-decorating gels. Red No 3 (CAS number: 16423-68-0; molecular weight: 879.86 g/mol) is an organoiodine compound. It is the disodium salt of 2,4,5,7-tetraiodofluorescein. FD&C Red No. 40 (Allura Red; CAS number: 25956-17-6; molecular weight: 496.42 g/mol)) is a dark red azo dye used in sports drinks, candy, condiments, and cereals. It is usually provided as its red disodium salt, soluble in water.

### Superabsorbent Polymer (SAP)

Granular Superabsorbent Polymer (SAP) particles were obtained from the extraction of SAP powder from the commercial Huggies® panty (Kimberly-Clark Company, purchased in Korea). SAP particles from the extraction were a full cut-size fraction of approximately 100 and 800 microns, indicating most of the sized fraction was within 300 and 500 μm.

### Methods

### Treatment Of Spherical Activated Carbon Particles

Examples of Treatment or Preparation Methods have Designations by Alphanumeric Numbers.

### A. Acid or Base Treatment

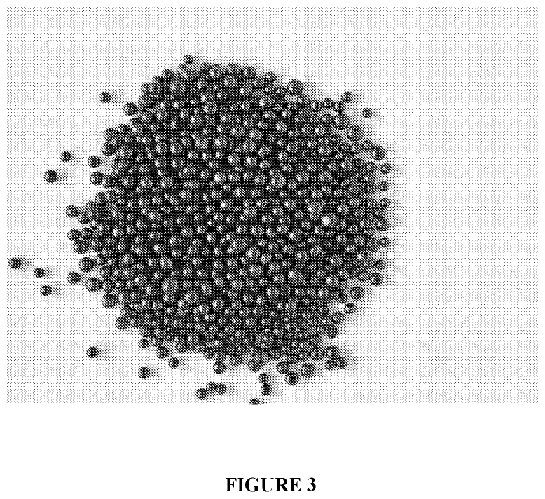

Typically, 50 g of spherical activated carbon particle materials from Kureha Corporation (see FIG. 3) are placed in a stainless-steel mixing bowl. A varying amount of acid or base, for example, citric acid solution (about 50 wt. % based on the water) or sodium silicate solution (about 40 wt. %), was added slowly under agitation, which was mixed readily and formed a wet slurry when mixed. The mixture was then dried in an air-circulated oven at about 80° C. for about 30 minutes. At the drying time of 15 minutes, the citric acid-coated particles were very lightly agglomerated, but the dried bead particles after 30 minutes were well free-flowing without showing any agglomerates.

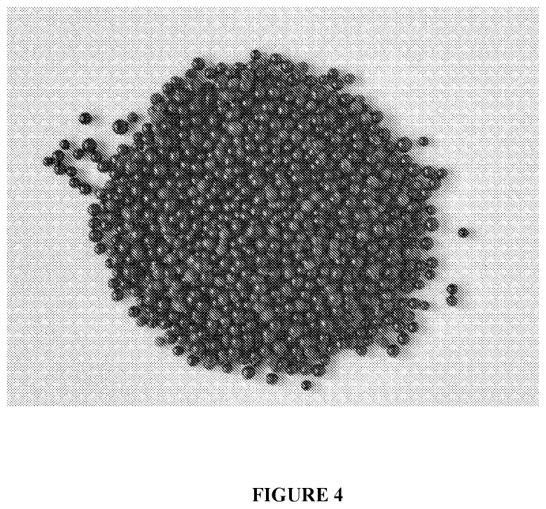

Throughout the Examples, it was found that citric acid coating of the spherical activated carbon particle is a reproducible process that can be readily processed. (FIG. 4) The high solubility of citric acid makes it easy to add a relatively more elevated amount of citric acid to the bead particle surfaces.

The sodium silicate solution was distributed well and homogeneously when added to the spherical activated carbon particles. In the treatment with spherical activated carbon of grade A-BAC-MP, a few wet agglomerates resembled raspberries in morphologies and were mainly composed of about 1-2 mm clumps. After drying, those agglomerates became fewer and were of several hundreds of μm to 1-2 mm, indicating sufficient bindings between the particulates. The slight agglomeration suggests the need for adequate mixing during the process.

### B. Red Color Treatment

The red color-activated composition was prepared, wherein the red color used was an artificial water-soluble food color (McCormick's Food color Red). About 2 g of spherical activated carbon (G-BAC-G70R) (FIG. 3) was placed in a 50 mL glass cup and added with about 10 droplets of red color. The red color was well distributed onto the particle surfaces. While adding the red color to the bead particles, the particles were not sticky to each other. The mixture was then placed in an air-circulated oven set at about 80° C. for 60 minutes.

### C. Candelilla Wax Treatment of Activated Composition

Melt coating of Candelilla wax is conducted as follows: flakes of virgin Candelilla wax (chopped and sifted through <500 μm) were used for melt coating of the spherical activated carbon particles containing citric acid. First, 10 g of activated composition (or spherical activated carbon particles such as G-BAC-G70R grade) were placed into a glass cup in the water bath and gradually heated. When the content reached about 70-75° C., 1.1 g of the chopped wax was added to acid-coated spherical particles, and the mixture was mixed thoroughly by hand using a wooden bamboo spatula for a further 3-5 minutes. It was found that the warm mix spread well on wax paper and made it easier to gather the individual particles and some small amount of agglomerates upon cooling down to room temperature.

### D. Preparation of Essential Oil Containing Melting-Point Depressed Candelilla Wax

Approximately 3.1 g of virgin Candelilla wax flakes were added to an aluminum container placed in a hot water bath (about 85˜90° C.). Within a few minutes, the wax gave a homogenous and transparent melt. Then, about 6.2 g of MCT oil and about 0.8 g of lavender essential oil were added. Lavender oil and MCT oil are miscible, and the mixture was readily homogenized and transparent, which was then cooled down to room temperature. For better comparison, a melting point depressed Candelilla wax via MCT oil was prepared with no lavender oil, otherwise prepared in the same way described above, wherein the melting point range was determined to be about 45-47° C. (Modified Wax A). The melting point of the Candelilla wax, MCT oil, and Lavender oil was about 42-44° C. (Modified Wax B).

### E. Preparation of Melting-Point Depressed Candelilla Wax Dispersion Comprising Essential Oil (Wax Dispersion A)

An aqueous dispersion of melting-point depressed Candelilla wax was prepared in the presence of a co-emulsifier such as Polysorbate-80 (Tween-80) in addition to a soy lecithin emulsifier. First, about 3 g of the melting-point depressed Candelilla wax with lavender oil prepared, as described in the Starting Example D, was molten and added with about 100 mL of boiling water, including about 0.6 g of soy lecithin powder and about 0.6 g of Polysorbate-80. The mixture was agitated with a high-speed kitchen hand mixer for one minute, and then about 0.15 g of TiO₂ powder was added and continued stirring for an additional couple of minutes. The mixture with approximate solids of 4 wt. % gave a very stable and milk-like dispersion. Next, the aqueous Candelilla wax dispersion was cooled to room temperature. The dispersion gave only a low-intensity delicate lavender smell. The color of the dispersion is still very white and milky. The wax dispersion A was free-flowing, and no signs of coagulation on the walls of the container bottle were seen, nor sediment for over 6 months.

### F. Preparation of Candelilla Wax Dispersion (Wax Dispersion B)

An aqueous dispersion of virgin Candelilla was prepared as follows: First, about 10 g of molten Candelilla wax and about 100 mL of boiling water were agitated with a high-speed kitchen hand mixer for about 2 minutes in the presence of about 2 g of soy lecithin powder, 2 g of Polysorbate-80, and 0.4 g of TiO₂, followed by mixing with a high-speed kitchen hand mixer for an additional 2-3 minutes. The final aqueous Candelilla wax dispersion with an approximate 12.6 wt. % solids was cooled down to room temperature and gave a thick-milky stable dispersion of beige color. The wax dispersion B was free-flowing, and no signs of coagulation on the walls of the container bottle were seen, nor sediment for over 6 months.

### G. Candelilla Wax Dispersion Treatment of Citric Acid-Activated Composition

An aqueous dispersion of Candelilla wax (Wax Dispersion B) prepared in Starting Example F was used for surface coating of the citric acid-activated composition particles. Typically, about 10 g of acid-coated particles were placed into a glass cup at room temperature, about 3 g of the aqueous Candelilla wax dispersion was added to acid-treated spherical particles, and the mixture was mixed thoroughly by hand using a wooden spatula for a further couple of minutes. Next, the mixture was spread onto wax paper and air-dried at room temperature. There was no sign of aggregates.

### H. Candelilla Wax Treatment of Spherical Cellulose Particles

The melt coating of spherical cellulose particles, Cellets 1000, is conducted as described in the Starting Example C above. First, about 10 g of Cellets 1000 particles were placed into a glass cup in the water bath and gradually heated the glass cup. When the content reached about 50-55° C., 1.1 g molten wax of the melting point depressed wax without lavender oil (Modified Wax A) described in Starting Example D above was added to Cellulose particles. The mixture was mixed thoroughly by hand using a wooden spatula for a few minutes. The resulting spherical cellulose particles were slightly cohesive upon cooling to room temperature. There was no sign of aggregates.

### I. Aqueous Dispersion of Candelilla Wax Treatment of Spherical Cellulose Particles

An aqueous dispersion of virgin Candelilla wax (Wax Dispersion B) from Starting Example G was used to surface coat of the spherical cellulose particles. Typically, about 10 g of Cellets 1000 were placed into a glass cup at room temperature, about 1.5 g of the aqueous Candelilla wax dispersion (Wax Dispersion B) was added to Cellulose particles, and the mixture was mixed thoroughly by hand using a wooden spatula for about 3 minutes. Next, the mixture was spread onto wax paper and air dried at room temperature. The Wax Dispersion B treated Cellulose particles were almost dry-looking and free-flowing individual particles with little cohesiveness. There was no sign of aggregates.

### Testing Procedures for the Properties of Composition of the Examples

The following test methods are used to describe specific properties of various components or articles of this invention.

### Sniff Testing—Odor Control Efficacy Testing

A sniff testing was performed to evaluate malodor control efficacy. A 500 mL volume glass jar was used as the sniff testing bottle. The bottles and sample sat at room temperature for several hours before being sampled. An amount of 0.25 g of dry samples in a small PP (polypropylene) container of approximately 30 mL of volume with a top diameter of 5 cm, a bottom diameter of 3.8 cm, and a height of 3 cm was placed into the 500 mL test glass jar with an airtight screwable lid. In addition, each jar sample was equipped with a small shallow PP plastic tray with a top diameter of 5 cm, a bottom diameter of 4.6 cm, and a height of 3 mm in which about 0.5 g of omega-3 oil or Kimchi broth was placed.

Sniff testing was performed at specified intervals at room temperature for over 24 hours, using the following scores
0=non-malodorous; 1=slightly malodorous; 2=malodorous; 3=strongly malodorous.
   The sniff samples were typically prepared in 5 to 7 samples. However, in some cases, the sample number was kept below 4. The sniff test was performed by an experienced person and repeated a few times at each interval. The average was rounded up or down to the next 0.5 unit for better comparison. The sniff testing conducted using the same materials simultaneously and at different times showed reproducibility.

### Candelilla Wax Melting Point Determination

In the present invention, Candelilla wax was tested for melting point. First, each wax sample or mixture in a small 50 mL aluminum container was placed in the hot water bath (85˜90° C.). Then, a probe thermometer measured the melting wax and its temperature until the mixture became clear and honey-like. When the mixture was molten, the mixture was taken out of the hot water bath, and the test container was placed on a dry paper towel; the mixture was observed by measuring the temperature as the mixture was cooling at the ambient temperature without forced cooling. The molten mix first showed a homogenous appearance, clear, transparent, and amber-yellowish. Then, the temperatures were taken at the melting point range when a thin yellowish-beige skim-fat-like surface skins formed, while the temperature decrease was slowed down or kept within a narrow range for a few minutes.

Candelilla wax flakes were melting at approximately 65 to 69° C. The melting temperature of the wax, in general, is difficult to determine due to the non-crystallin nature of the material. At 0 wt. % MCT (virgin Candelilla wax) based on total weight, a melting point range of 66-70° C. was determined; at 17.8 wt. % MCT, 57-62° C.; at 30.9 wt. %. 52-56° C.; at 42.3 wt. %, 49-54° C.; at 55.6 wt. %, 46-51° C.

Vaseline® is odorless, tasteless, and slightly yellowish or white, a soft jelly-like mass (petroleum jelly), and was found to have a melting point range of 27-36° C. according to the present method.

The melting point data is not absolute and thus needs to be taken as a qualitative measure. However, despite the qualitative data, the melting point depression grew systematically as the MCT amount increased, which is a valuable result applicable to various products.

### Schlieren Test of Coated Particles via Water Contact

The schlieren phenomenon is observed when there are density inhomogeneities in transparent fluids. The present invention observed a downward stream (schlieren) of acid and red color due to the density differences against the pure water. The schlieren phenomenon observed using an LED magnifying lamp was depicted as the colorless citric acid stream or contrast-rich red color when activated composition particles (typically, 10-20 spherical particles) were put onto the water surface. The schlieren phenomenon in this invention was shown as the citric acid or red color dissolved in water in a filled glass cup and migrating from the spherical particle surfaces.

### Direct pH Test of Acid Coated Particles

The surface acidity and basicity are directly seen via a pH strip test. For example, citric acid or sodium silicate-activated composition particles turn the color red (acidic), deep green (lightly basic, e.g., baking soda), or deep purple (sodium silicate) on the wet pH paper instantly and indicate an apparent surface acidity or basicity of the particles

### Amine Testing via Draeger™ Tube

Draeger short-term gas detection tube, the amine tubes (part number 8101061, qualitative, color change from yellow to blue) was used to determine the presence of an amine. The tube is qualitative testing, changing color from yellow to blue in the presence of amines at ambient temperature. The tube's amine sensitivity is relatively low, so gases reacting basic shall cause the color change unspecifically. In the present invention, the tube ends were broken off and placed into the sniff testing jar for several days in the presence of marine omega-3 oil (Control A).

### Shaking Experiment of Granular SAP Particles and Spherical Activated Carbon Particles

About 10 g of the granular SAP particles and about 1 g of spherical activated carbon particles, G-BAC-70R, were placed in a 100 mL glass jar with a screwable metal lid and shaken by hand for 20 minutes at an approximate 50 to 60 Hz. After 20 minutes, the mixture was examined for possible contamination by black specks of dust and/or black particles. The empty bottle wall was also examined for possible signs of contamination.

### General Procedures

In the following Examples, the lettered Examples are Comparative, and the numbered Examples are this invention.

Control samples A and B illustrate marine omega-3 oil representing a complex fishy smell and Kimchi broth representing a complex smell of sulfur-containing volatile organic compounds (VOCs), including volatile aldehydes and acids.

Comparative Examples. Examples A and B, illustrate activated carbon powder to represent a current state-of-the-art, carbon-type malodor control material, and sodium bicarbonate (baking soda) powder to represent a current state-of-the-art, non-carbon-type malodor control agent.

Examples 1 to 4 illustrate various non-treated and acid or base-activated composition particles, wherein the non-treated activated composition, Example 1, is Kureha grade A-BAC-MP, and Examples 3 and 4 serve as reproducibility studies for the efficacy of sodium silicate treatment.

Examples 5 to 8 illustrate various non-treated and acid or base-activated composition particles, wherein the non-treated activated composition, Example 5, is Kureha grade G-BAC-G70R.

Example 9 illustrates a mix comprising 56 wt. % of Example 8 and 44 wt. % of Example 6 based on the total mixture weight.

Example 10 illustrates a red color-activated composition.

Examples 11 and 12 illustrate Candelilla wax- and an aqueous dispersion of Candelilla wax (Wax Dispersion B) treatment to citric acid-activated composition (Example 6), respectively.

Examples 13 and 14 illustrate Candelilla wax- and an aqueous dispersion of Candelilla wax treatment (Wax Dispersion B) to red color-activated composition (Example 10), respectively.

Example 15 illustrates a block of Candelilla wax with a depressed-melting point and Essential oil (Modified Wax B).

Example 16 illustrates the aqueous dispersion of Candelilla wax with a depressed-melting point and Essential oil (Wax Dispersion A).

Example 17 illustrates the spherical cellulose particles treated with melt-coating of Candelilla wax (Modified Wax A).

Example 18 illustrates the spherical cellulose particles treated with an aqueous dispersion of Candelilla wax (Wax Dispersion B).

Example 19 is a mixture of irregular-shaped commercial granular SAP particles and spherical activated carbon particles, illustrating the attrition behavior of the mixture.

Examples 20 illustrates non-treated spherical activated carbon particles, PureSphere grade PureCarbon L.

### Examples 1 to 8

### Citric Acid or Sodium Silicate Treatment of Spherical Activated Carbon Particle

In Table 1, the acid- and base-activated compositions of the present invention are shown. The spherical activated carbon particle samples were prepared using citric acid or sodium silicate solution, using two different types of non-treated activated composition particles (Kureha A-BAC-MP and G-BAC-G70R).

TABLE 1
Acid and Base Treatment of Spherical Activated Carbon Particle

| | A-BAC-MP | | G-BAC-G70R | |
|---|---|---|---|---|
| | The citric | Sodium | The citric | Sodium |
| | acid | Silicate | acid | Silicate |
| Treatment | (wt. %)¹ | (wt. %) | (wt. %) | (wt. %) |
| Example 1 | N/T² | N/T | — | — |
| Example 2 | 56 wt. % | — | — | — |
| Example 3 | — | 40 wt % | — | — |
| Example 4³ | — | 40 wt. % | — | — |
| Example 5 | — | — | N/T | N/T |
| Example 6 | — | — | 25 wt. % | — |
| Example 7 | — | — | 60 wt. % | — |
| Example 8 | — | — | — | 44 wt. % |

| | | | |
|---|---|---|---|
| Example 9⁴ | — | — | a mixture of 56 wt. % of Example |
| | | | 8 and 44 wt. % of Example 6 |

¹Based on the weight of the activated composition weight
²N/T means Non-treated spherical activated carbon particle
³Reproducibility
⁴Example 9 comprises 56 wt. % of Example 8 and 44 wt. % of Example 6 based on the total mixture weight

Table 1 shows citric acid and sodium silicate solution treatment of spherical activated carbon particles and a physical blending of the acid- and base-activated composition. Both solutions were mixed readily and formed a wet but good agitable blend After drying, the citric acid-coated particles were already free-flowing individual particles (FIG. 4), indicating the good thermal conductivity of the spherical activated carbon material. The citric acid treatment of the spherical activated carbon is a reproducible process that can be readily processed. The high solubility of citric acid is beneficial to add citric acid at an elevated amount to the particle surfaces.

Sodium silicate solution was distributed well and homogeneously when added to the spherical particles. As a result, flowable, individual activated composition particles are obtained. A small number of agglomerates were found in the sodium silicate treatment of A-BAC-MP, resembling raspberries in morphologies. A strong force was needed to break up the agglomerates by hand to break up the bonding between the bead particles. Also, the particles showed signs of both interparticle and intraparticle break-ups, which indicated that the bonding was strong enough to mechanically fracture the bead structure, a finding that was reproducible as confirmed in Examples 3 and 4. The results suggest that the sodium silicate treatment agglomerates with stronger bonding. Therefore, a more effective means of agitation may be required to avoid agglomerating completely.

### Control A Vs. Example 1 and Example 2

### Complex Fishy Smell Malodor Control Efficacy of Spherical Activated Carbon Particle and Acid-Activated Activated Composition

Control A was tested against a non-treated and a citric acid-activated composition particle, Example 1 and Example 2, respectively, and subject to a sniff test for liquid Omega-3 oil for 45 hours. In Control A, marine omega-3 oil was used to represent the complex fishy smell. The sniff test results are summarized in Table 2

TABLE 2
Complex Fishy Smell via Marine Omega-3
Oil Malodor Control Efficacy Testing

| | Time (Hour) | | | | | | | |
|---|---|---|---|---|---|---|---|---|
| | 1 | 2 | 3 | 4 | 6 | 8 | 24 | 45 |
| Control A | 3 | 3 | 3 | 3 | 3 | 3 | 3 | 3 |
| Example 1 | 2 | 2 | 2 | 2 | 2 | 2 | 1.5 | 1.5 |
| Example 2 | 2 | 1.5 | 1.5 | 1.5 | 1 | 1 | 1 | 1 |

Table 2 shows malodors detected by olfactory evaluation over time. Example 1 and Example 2 showed a significant malodor control efficacy against Control A, which is the complex fishy smells derived from marine omega-3 oil. In addition, when Control A was sniffed, a powerful fishy smell was noticed throughout the testing period, resembling the complex fishy smells in feminine napkins, menstruations, and vaginal discharges.

Control A was subjected to an amine test via Draeger short-term gas detection tube. The tube did not change color, indicating the absence of amines or other gases that might react. The result from this amine testing is surprising because, in the art, the fishy smell usually is associated with amines and/or ammonia-type basic gases. For example, trimethylamine (TMA) has been used frequently to simulate the fishy smells in feminine napkins and light incontinence applications in the art. Therefore, the observations made in the present invention indicate that the complex fishy smells derived from the oxidative reactions from marine omega-3 oil may be free of amines and thus represents the complex interplay of malodors and distinct malodor compounds effectively.

Table 2 also shows clearly that the acid-treated Example 2 has higher malodor control capability than Example 1 and is superior to Control A. The results suggest that the acid treatment of the spherical activated carbon of the present invention is a highly effective malodor control agent against the complex fishy smell derived from marine omega-3 oil over a period greater than 24 hours.

### Control B vs. Example 1 and Example 2

### Kimchi Malodor Control Efficacy of Acid-Activated Composition

Control B, in which Kimchi broth was used only as a control, was tested against a non-treated activated composition (Example 1) of the present invention and citric acid-activated composition (Example 2), wherein the sniff test was performed for 45 hours. The sniff test results are summarized in Table 3.

TABLE 3
Kimchi Malodor Control Testing

| | Time (Hour) | | | | | | | |
|---|---|---|---|---|---|---|---|---|
| | 1 | 2 | 3 | 4 | 6 | 8 | 24 | 45 |
| Control B | 3 | 3 | 3 | 3 | 3 | 3 | 3 | 3 |
| Example 1 | 2 | 2 | 2 | 2 | 2 | 2 | 2 | 1.5 |
| Example 2 | 2 | 1.5 | 1.5 | 1.5 | 1.5 | 1.5 | 1.5 | 1.5 |

In Example 1, the non-treated activated composition shows high efficacy against strongly malodorous Kimchi broth. Example 2, the citric acid-activated composition particles reduced significantly adverse malodors from Kimchi over 45 hours. It also becomes evident that Example 1 of the present invention still shows high efficacy in controlling the pungent Kimchi malodors (Control B).

### COMPARATIVE EXAMPLES

### Control B, Examples A and B vs. Examples 5, 8, and 9

### Kimchi Malodor Control Efficacy of Sodium Silicate Activated Composition

In Table 4, the evaluation of two comparative Examples for Kimchi malodor control efficacy, commercially available activated carbon powder (Comparative Example A) and sodium bicarbonate (baking soda) powder (Comparative Example B), respectively, is shown. Table 4 further indicates the malodor control efficacy of the particles of non-treated activated composition (Example 5) and sodium silicate activated composition (Example 8) against Kimchi malodors. Additionally, Table 4 shows the efficacy of Example 9 (Table 1), representing a simple physical blend comprising 44 wt. % of an acid-activated composition (Example 6) and 56 wt. % of a base-activated composition (Example 8) based on the total mixture weight, wherein citric acid and sodium silicate was the acid and base.

TABLE 4
Malodor Control Efficacy against Kimchi
Malodors Control Testing

| | Time (Hour) | | | | | | |
|---|---|---|---|---|---|---|---|
| | 1 | 2 | 4 | 6 | 8 | 27 | 52 |
| Control B | 3 | 3 | 3 | 3 | 3 | 3 | 3 |
| Example A | 2 | 2 | 2 | 2 | 2 | 2 | 2.5 |
| Example B | 2 | 2 | 2 | 2 | 2 | 2 | 2.5 |
| Example 5 | 2 | 2 | 2 | 2 | 2 | 2 | 2 |
| Example 8 | 2 | 1.5 | 1 | 1 | 1 | 1 | 1 |
| Example 9 | 2 | 1.5 | 1.5 | 1.5 | 1.5 | 1.5 | 1 |

It is seen that the activated composition of the present invention effectively controlled the complex Kimchi malodors. Surprisingly, the sodium silicate activated composition was very effective from early until over 50 hours. On the other hand, activated carbon powder (Example A; FIG. 1) showed consistent malodor control efficacy. Still, it did show a lower efficacy than that of the activated compositions of the present invention.

Furthermore, baking soda (Example B; FIG. 2) has been widely used as an effective odor control agent, and it is also effective against Kimchi malodors, as seen in the results. However, the results were still not as effective as those of the activated compositions of the present invention.

Both activated carbon and baking soda powder used as controls are fine powders, and handling and use are cumbersome. At the same time, the present invention provides an activated composition with higher efficiency with easy handling.

In Table 4, Example 9 suggests that a physical blending of the acid- and base-activated composition may still be effective. Such blending would allow an effective and flexible design of the articles focused on the types of malodors.

### Control A, Example A and B vs. Examples 1.2, 5, and 7

### Complex Fishy Smell Malodor Control Efficacy of Citric Acid-Activated Composition

TABLE 5
Complex Fishy Smell Malodor Control Efficacy
of Citric Acid-Activated Composition

| | Time (Hour) | | | | | | |
|---|---|---|---|---|---|---|---|
| | 1 | 2 | 3 | 4 | 6 | 16 | 41 |
| Control A | 3 | 3 | 3 | 3 | 3 | 3 | 3 |
| Example A | 2 | 2 | 2 | 2 | 2 | 2 | 2.5 |
| Example B | 2 | 2 | 2 | 2 | 2 | 2.5 | 2.5 |
| Example 1 | 2 | 2 | 2 | 2 | 2 | 2 | 1.5 |
| Example 5 | 2 | 2 | 2 | 2 | 2 | 2 | 1.5 |
| Example 2 | 2 | 2 | 1.5 | 1.5 | 1 | 1 | 3 |
| Example 7 | 2 | 2 | 1.5 | 1.5 | 1 | 1 | 1 |

Table 5 shows that the non-treated activated composition of the present invention, Example 1 and Example 5, irrespectively to the citric acid treatment, controlled the complex fishy smell malodors more effectively than Comparative Example A (commercial activated carbon powder) (FIG. 1) and Comparative Example B (sodium bicarbonate/baking soda) (FIG. 2) for about 40 hours.

Noteworthy is that both activated carbon powder (Example A) and sodium bicarbonate powder (baking soda; Example B) show consistent malodor control efficacy, although they don't reach the level of the current invention's activated compositions. Both Examples A and B per se are not ineffective methods, as can be seen in Table 5 and the previous Tables. However, as mentioned earlier, handling and using large quantities of activated carbon (Example A) powder and baking soda (Example B) powder is cumbersome to process and manufacture the final consumer products, in addition to those many other issues already described.

Table 5 also shows clearly that the acid-treated Examples 2 and 7 have higher malodor control capability than Example 1 and 5 and is superior to Examples A and B irrespective of the particle sizes of the spherical activated carbon of the present invention.

The present invention provides an activated composition with easy handling and excellent efficacy.

### Summary of these Above Results

The results from the preceding Examples suggest that the present invention's activated composition represents an effective means for malodors, which are complex, such as complex fishy smell not comprising amines and sulfur-containing VOCs.

The activated composition of the present invention is superior to the state-of-the-art coconut shell-based activated carbon powder, or baking soda powder, not only in their efficacy of malodor control capabilities but also in various processibility characteristics compared to the latter. These include high-fill capability and capacity due to the increased bulk density, high flowability due to the spherical shape, extremely low carbon dust, high purity, high strength, high wear resistance, and narrow particle size distribution and controllability of the particle sizes from sub-microns to a few mm sizes.

In addition, it is noteworthy that high purity is beneficial for applications such as personal hygiene products and oral, medical, and smokeless tobacco use in people's mouths, among others.

Furthermore, the particle sizes of the present invention can also be prepared to be the right size ranges when used in the person's mouth regarding a smoother mouth feel.

The activated composition of the present invention is characterized by spherical geometry. Therefore, it has the potential as an effective malodor control agent when used with cellulose fibers in absorbent cores for hygienic applications.

The spherical activated composition with smooth surfaces and non-dusty properties can be more readily incorporated into the absorbent core via effective anchoring in the microcavities formed by the cellulose fluff fibers; it may not produce pinholes due to the absence of the sharp edges, wherein the pinholes in the thin back sheet film of hygiene articles are attributed not only to leading cause for potential leakages of urine but also an uncontrolled release of malodors through the pinholes; may help reduce potential VOCs inside the absorbent articles; may have the real potential to introduce a malodor control agent in the absorbent article without dust issues.

### Example 10

### Preparation of Red Color-Activated Composition

Example 10 is obtained by treating the spherical activated carbon particle (G-BAC-70R grade) with red color, as described in the Treatment Example B. Example 10 comprises all free-flowing individual particles and is non-sticky and shiny as their starting bead material. They showed no signs of coagulation or aggregation.

### Example 11 to 14

### Candelilla Wax Treatments for Acid and Red Color-Activated Composition

In Table 6, Examples 11 to 14 were prepared using the spherical activated carbon particle (G-BAC-G70R grade). Examples 11 and 12 were prepared using Example 6, wherein Example 6 is an acid-activated composition containing about 25 wt. % citric acid based on the weight of the activated composition. Examples 13 and 14 were prepared using Example 10, the red color-activated composition. Examples in Table 6 are subject to the schlieren test, and the results are summarized below

TABLE 6
Candelilla Wax Treatments for Acid-
and Red Color-Activated Composition

| | Example | Example | Example | Example |
|---|---|---|---|---|
| | 11 | 12 | 13 | 14 |
| Activated | Example 6 (Citric | | Example 10 (Red | |
| Composition | acid Treatment) | | color Treatment) | |
| Candelilla | Dry Wax | Aqueous Wax | Dry Wax | Aqueous Wax |
| Wax Type | | Dispersion | | Dispersion |

1) Based on the total weight of the aqueous dispersion

### Schlieren Test of Citric Acid-Activated Composition Particles

Example 6, containing 25 wt % citric acids, showed an obvious sign of schlieren: as soon as the acid-treated particles contacted the water, an instant stream of schlieren happened without exceptions. However, both Examples 11 and 12 showed no indications of schlieren.

pH test

An apparent acidity was seen when individual particles of Example 6 were tested for pH. Furthermore, when a bunch of Example 6 particles was placed on the wet pH strip, an instant color change of the pH strip from every single particle was observed, showing a pH of about 4-5. On the other hand, Examples 11 and 12 did not show pH color changes: No color changes were seen even after several hours and until the wet pH paper was dried out at air temperature. The results suggest that the wax materials tightly shielded the migration of the acidic compound.

The results from the schlieren and the pH color change suggest that the Candelilla wax coating on the acid-activated composition particles has performed well.

### Schlieren Test of Red Color-Activated Composition

Example 10, red color-activated composition particles, observed using an LED magnifying lamp, showed a remarkably apparent schlieren phenomenon in the water: the red color instantly dissolved in water, and the intensive red color stream was depicted in the form of the schlieren phenomenon, which was thanks to the color readily visualized. In Example 10, without exceptions, all particles instantly showed strong streams of red color schlieren phenomenon.

Individual particles still showed clear signs of the schlieren phenomenon, even after 5-7 minutes. After exhaustion, the schlieren phenomenon is weakened. The extent how fast the red color is migrating out of the particles may be the qualitative indication of the leachable red color on the surface. It was found, however, that the reddish color of the total volume of the water in the glass cup (water color) is an indicator of the cumulative total red color migration of given particles over several hours.

### Schlieren Test of Candelilla Wax Coated Red Color-Activated Composition

In Examples 13 and 14, wherein melt or aqueous dispersion of Candelilla wax (Wax Dispersion B) was used to encapsulate Example 10, a stream was significantly weaker than Example 10. The weak red color stream even disappeared altogether after a short time of about 1 to 2 minutes. In Examples 13 and 14, the watercolor was weak after 8 hours and looked like almost clear water. The weak color streams at the beginning are believed to be dyes captured between interparticle spaces in small quantities.

The results suggest that both wax coating methods, melt or aqueous dispersion, effectively shielded the migration of the red color from the activated composition particles. The results are compared with those from Examples 10 and 11.

### Hot Water Testing for Examples 13 and 14

Some particles of Examples 13 and 14 were taken out from the water from the glass cup after 8 hours of standing. Then, those particles were placed back into the test glass cup with hot water of about 80-85° C. These particles from Examples 13 and 14 showed fresh and remarkably intensive red color streams again, indicating that the color was well encapsulated until the wax layer was molten.

In another test, Examples 13 and 14 were freshly immersed in hot water at about 80-85° C. At first, particles from Examples 13 and 14 showed the weak schlieren briefly at the water contact. Then, the immersed particles showed an explosively intensive and robust stream of red color. The result indicates that the wax layer encapsulated tightly and hindered the red color's migration out of the particles.

It is believed that the melting-point-depressed wax of the present invention may control the release of the actives at a lower and desired temperature in diverse applications, such as the temperature of the mouth and vulvar area.

### Implications of Red Color Results for Release of the Actives

It is important to note that the red color is a demonstrative example of many possible active agents disclosed in the present invention.

According to McCormick, the primary two FD&C colors in the used food red are soluble FD&C Reds 40 and 3 and have molecular weights of about 496.4 and 879.8 g/mol, respectively. Also, the red dye is a salt form representing the sufficiently polar nature of the red color compounds. Nicotine (CAS number: 54-11-5) has a molecular weight of about 162.2 g/mol. Caffeine (CAS number: 58-08-2) has a molecular weight of about 194.2 g/mol. The primary compound of Cannabis is tetrahydrocannabinol (THC) (CAS number: 1972-08-3), with a molecular weight of about 314.5 g/mol. As can be seen, the probe molecules in the red color used in the present invention are more significant in molecular weight and size than those from nicotine and nicotine salt, caffeine, tetrahydrocannabinol, and a variety of the active release agents of the present invention such as flavonoids, flavors, and essential oils, among others.

The overall red color results indicate the activated composition's excellent adsorption and desorption behavior, which can be controlled via wax encapsulation and temperature. In addition, the preceding Examples demonstrate the amphiphilic nature of the activated composition particles. Combined with high surface areas, the activated composition of the present invention may provide many charged and non-charged, polar and non-polar; hydrophobic and hydrophilic compounds; and small and relatively large molecular weight compounds to be accommodated.

### Examples 15 and 16

### Encapsulated Essential Oil in Aqueous Dispersion of Candelilla Wax (Wax Dispersion A)

Example 15 is composed of a depressed melting point Candelilla wax containing lavender oil as Essential oil and was prepared as described in the Starting Example D.

Example 15 gave a substantially weaker lavender smell than the liquid mixture of MCT oil and lavender oil, indicating a reduced lavender oil release by merely being incorporated in the wax.

Example 16 is an aqueous dispersion of the Candelilla wax with a depressed melting point (Wax Dispersion A) and Essential oil (Example 15) and was prepared as described in the Starting Example E. In addition, the wax dispersion A was free-flowing and could be sprayed by a simple sprayer without causing coagulation issues.

Both Examples 15 and 16 were stored in the fridge overnight at about 4° C. After that, both Examples were kept for an hour and a half at room temperature before taking a sniff testing, compared with the smell of the lavender of a liquid oil mixture of lavender and MCT composed of the same concentration as Examples 15 and 16.

Example 16 gave a delicate lavender smell, which was even weaker than that of Example 15.

The results indicate that lavender oil is encapsulated effectively by both wax forms, for example, the thin wax melt or the finely divided wax microparticles with a depressed melting point dispersed in an aqueous phase. The result also implies that the wax encapsulation may substantially delay and/or control the release of volatile compounds, which implies significant advantages, for example, in nicotine pouches use.

### Examples 17 and 18

### Spherical Cellulose Particles and Candelilla Wax Treatment

Examples 17 and 18 exemplify wax-coated spherical cellulose particles. The spherical cellulose particles employed in the present invention are Cellets 1000 with particle sizes between about 1000 μm to 1400 μm.

Example 17 comprises the spherical cellulose particles treated with melt-coating of Candelilla wax by melt coating using the Starting Example D without lavender oil.

Example 18 comprises the spherical cellulose particles treated with an aqueous dispersion of Candelilla wax. (Wax Dispersion B).

The result shows the process feasibility of the wax treatments on cellulose spheres.

The results also suggest that a spherical cellulose particle containing actives such as nicotine, caffeine, cannabinoids, and other actives disclosed in this application, might be used in a mixture with the activated composition of the present invention.

Further, the exact spherical geometry, controllable sizes, and surface areas of cellulose spheres and activated composition spheres may make the effective release of the actives more versatile for various applications.

### Example 19

### Attrition Behavior of Granular SAP Particles and Spherical Activated Carbon Particles

Example 19 is a mixture of irregular-shaped commercial granular SAP particles and non-treated spherical activated carbon particles, which was prepared to determine the relative attrition behavior of SAP particles to that of the activated composition of the present invention, wherein the mixture comprises about 91 wt. % SAP and about 9 wt. % spherical activated carbon particles based on the weight of the total mixture.

The SAP particles did not show any signs of contamination by black dust or small carbon particles, which indicates no attrition from the spherical activated carbon particles. The activated carbon particles also showed no signs of fractures and/or no loss of shiny appearance. In contrast, the SAP particles showed attrition due to the mechanical impact, resulting in some fine particles (“fines”), which were readily visible and could be gathered.

This result suggests robustness and attrition resistance of the spherical activated carbon particles of the present invention: even irregular, angular, and edgy-shaped SAP granules could not cause physical damage.

The above results also imply that the robust spherical activated carbon particles may be utilized even in the current irregularly shaped granular SAP particles on a large process scale.

In addition, it is known that large-sized irregular-shaped SAP particles such as greater than about 700 μm to 800 μm create more fines particles while posing more potential pinhole problems. Thus, using a size fraction below about 650 μm would help maximize the malodor control efficiency when used with the activated composition in the hygiene article while minimizing the fines dust and avoiding potential pin holes.

The results also imply that using spherical SAP particles, for example, bead- or round-shaped SAP particles, would be even more advantageous when used with the activated composition of the present invention.

### Example 20

Complex Fishy Smell Malodor Control Efficacy of Example 20 vs. Comparative Examples A and B and Example 5

Table 7 shows a comparison of the malodor control effectiveness of Example 20 to Example 5 (Kureha grade G-BAC-G70R, a non-treated spherical activated carbon particle) and two Comparative Examples A and B. A 45-hour sniff test was conducted using liquid marine Omega-3 oil to simulate a complex fishy smell, with Control A representing the fishy odor intensity. The results are summarized in Table 7 below.

TABLE 7
Complex Fishy Smell via Omega-3
Oil Malodor Control Efficacy Testing

| | Time (Hour) | | | | | | | |
|---|---|---|---|---|---|---|---|---|
| | 1 | 2 | 3 | 4 | 6 | 8 | 24 | 45 |
| Control A | 3 | 3 | 3 | 3 | 3 | 3 | 3 | 3 |
| Example A | 2 | 2 | 2 | 2 | 2 | 2 | 2.5 | 2.5 |
| Example B | 2 | 2 | 2 | 2 | 2 | 2.5 | 2.5 | 2.5 |
| Example 5 | 2 | 2 | 2 | 2 | 2 | 2 | 2 | 1.5 |
| Example 20 | 2 | 2 | 2 | 2 | 2 | 2 | 2 | 1.5 |

In Table 7, both Example 20 (non-treated activated carbon particles derived from polymer beads) and Example 5 exhibited high effectiveness in controlling malodors associated with complex fishy smells derived from omega-3 oil when compared to Control A. Throughout the testing period, Control A consistently emitted a strong fishy odor.

Another spherical activated carbon particle, the product grade PureCarbon L was obtained from PureSphere Co., LTD., South Korea. PureCarbon L particles exhibit spherical properties and appearances analogous to those depicted in FIG. 3 for BAC, showcasing a consistency in form and structure. These particles are created by polymerizing spherical polystyrene-based polymer beads and subsequently carbonizing and activating them. Similar to Kureha's BAC, PureCarbon L offers excellent wear and attrition resistance, reducing carbon dust generation and the risk of contamination.

The spherical activated carbon particles from PureSphere Co., LTD., Korea have an average particle size, with a weight fraction of smaller than 0.20 mm (5 wt. %) and equal to or greater than 0.2 to 0.6 mm (95 wt. %) PureCarbon L's specific surface area, determined using the BET method, falls within the range of 1000±100 m2/g. Additionally, PureCarbon L exhibits a packing density of 0.65±0.05 g/cm3, highlighting its uniform spherical nature.

Table 7 underscores the remarkable malodor control capabilities of Example 20 and Example 5, both of which surpasses Examples A and B. These findings demonstrate the effectiveness of spherical activated carbon particles in combating complex fishy odors from marine omega-3 oil, surprisingly even during extended periods exceeding 45 hours. The present invention introduces a user-friendly and highly efficient non-treated, spherical activated carbon composition.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter. Accordingly, this description is to be construed as illustrative only and is for the purpose of teaching those skilled in the art the general manner of carrying out the invention.

1. A composition comprising a spherical activated carbon particle having a sphericity greater than 0.6, and at least one active agent.

2. The composition of claim 1, wherein the spherical activated carbon particle comprises a particle diameter from about 0.01 to about 5000 μm.

3. The composition of claim 1, wherein the spherical activated carbon particle comprises a particle having a BET-specific surface area of about 100 to about 2500 m²/g.

4. The composition of claim 1, wherein the spherical activated carbon particle further comprises an iodine adsorption capacity of about 100 to about 2500 mg/g.

5. The composition of claim 1 comprising: (a) about 5 to about 99.99 wt. % of spherical activated carbon particles; and (b) about 0.01 wt. % to about 80 wt. % of at least one active agent, and (c) about 0.01 wt. % to about 10 wt. % water, based on the total weight of the composition.

6. The composition of claim 1, wherein the spherical activated carbon particle is derived from pitch, non-treated spherical activated carbon particles derived from ion exchange beads, or non-treated spherical activated carbon particles from granulation technologies.

7. The composition of claim 1, wherein the active agent is: water; an acid; a base; urea; a colloidal particle; an auxiliary odor control agent; an active release agent selected from the group consisting of a bioactive agent, surface active agent, medicinally active agent and/or hygienically active agent, and cosmetically active agent; stimulating agents selected from the group consisting of nicotine and its salts, caffeine, and cannabinoids; essential oil; flavoring agent; sweetening agent; flavonoids; or a combination of active agents.

8. The composition of claim 7, wherein the acidic compound comprises a pH of less than about 6, wherein the acid comprises one or more acids selected from the group consisting of hydrochloric acid, hydrobromic acid, boric acid, nitric acid, sulfuric acid, phosphoric acid, mono- and di-carboxylic acids, benzoic acid, cinnamic acid, methanesulfonic acid, ethanesulfonic acid, p-toluenesulfonic acid, salicylic acid, low molecular weight polycarboxylic acids smaller than 5000 g/mol, and a combination of such acids.

9. The composition of claim 8, wherein the carboxylic acid is selected from the group consisting of acetic acids, citric acid, formic acid, fumaric acid, itaconic acid, maleic acid, lactic acid, methyl fumaric acid, malic acid, methyl maleic acid, oxalic acid, salicylic acid, succinic acid, 2-methyl succinic acid, tartaric acid, phthalic acid, powder of apple cider vinegar, and powdered vinegar, wherein the carboxylic acid is from about 0.1 wt. % to about 80 wt. % based on the total acid activated composition weight.

10. The composition of claim 7, wherein the base compound comprises a pH greater than 8, wherein the base comprises one or more bases selected from the group consisting of sodium carbonate, sodium bicarbonate (baking soda), sodium silicate (water glass), sodium hydroxide, potassium hydroxide, ammonium hydroxide, calcium hydroxide, sodium monoxide, calcium oxide, aluminum hydroxide, cesium hydroxide, magnesium hydroxide, barium hydroxide, ferric hydroxide, pyridine, or quaternary ammonium hydroxide, and a combination thereof, wherein the base is from about 0.1 wt. % to about 80 wt. % based on the total activated composition weight.

11. The composition of claim 7, wherein active agent comprises at least one auxiliary odor control agent selected from the group consisting of: spherical cellulose particles as carriers treated for odor control; spherical activated carbon particles from granulation technologies; a cyclodextrin; a porous polymer adsorbent; a plant extract wherein the extract is a dry powder of one or more of green tea leaves, olive leaves, Yucca, Aloe, or Quillaja; chelating agents; natural and synthetic zeolites; a zeolite powder; an ion-exchanged with silver, chitosan, and a mixture thereof, borax; a peroxide; a permanganate such as sodium or potassium permanganate; a biocide; chelating agents; perfumes; various metals and metal compounds, or a mixture thereof and used in a concentration of from about 0.01 wt. % to about 25 wt. %, based on the weight of the total activated composition.

12. The composition of claim 7, wherein the colloidal particles are selected from the group consisting of micron-sized or nano-sized fine powders of silica as hydrophobic and hydrophilic fumed silica, colloidal silica, and precipitated silica; alumina, titania, zirconia, diatomaceous Earth; superhydrophobic aerogels selected from the group consisting of silica aerogels, carbon aerogels, cellulose aerogels, metal aerogel powders, cellulose nanofibers, cellulose nanocrystals, and a mixture thereof, wherein the colloidal particle is used in the concentration of about 0.01 wt. % to about 15 wt. %, is based on the total activated composition weight.

13. The composition of claim 7, wherein the active agent is a release agent that controls and triggers release by liquid contact or temperature change, or a combination thereof, wherein the release agent comprises a moiety selected from a bioactive agent; a surface-active agent; a medicinally and/or oral hygienically active agent; a cosmetically active agent; a stimulating agent such as nicotine and its salts, caffeine, a cannabinoid; a flavonoid, an essential oil; a flavor; or a mixture thereof.

14. The composition of claim 1, further comprising phase change materials (PCMs) as an active agent, selected from Candelilla wax, Beeswax, Carnauba wax, Chinese wax, lanolin (wool wax), shellac wax, spermaceti, Bayberry wax, Castor wax, Esparto wax, Japan wax, Jojoba oil, Ouricury wax, rice bran wax, soy wax, Tallow tree wax paraffin wax, microcrystalline wax, or a mixture thereof.

15. The composition of claim 14, wherein the phase change materials (PCMs) further comprises a reduced melting temperature of the PCM induced by adding a compatible hydrocarbon oil such as MCT (medium-chain-triglyceride), wherein the melting temperature of the PCM is reduced by at least 0.5° C. or greater from that of the virgin PCM.

16. The composition of claim 14, further comprising an aqueous dispersion of the PCM, wherein the aqueous dispersion has one or more emulsifiers selected from lecithin, polysorbate 60, polysorbate 80, Carbomer copolymer, sodium stearate, sodium lauryl sulfate, sorbitan monolaurate, sorbitan monooleate, sorbitan monostearate, sorbitan mono-palmitate, sorbitan sesquioleate, stearic acid, or a mixture thereof.

17. The composition of claim 14, wherein the phase change materials (PCMs) are applied to the spherical activated carbon particles or spherical cellulose particles in the form of a melt coating or an aqueous dispersion.

18. The composition of claim 1, comprising:
(a) about 5 wt. % to about 99.99 wt. % of spherical activated carbon particles comprising carbon derived from pitch or ion exchange beads having sphericity of at least 0.6, a particle diameter of about 0.01 μm to about 5000 μm, a BET-specific surface area of from about 100 to about 2500 m²/g, and an iodine adsorption capacity of 500 mg/g or greater;
(b) about 0.01 wt. % to about 10 wt. % of water;
(c) about 0.1 wt. % to about 80 wt. % of a treatment compound of an acid, base, or urea;
(d) about 0.01 wt. % to about 15 wt. % of a colloidal particle;
(e) about 0.01 wt. % to about 80 wt. % of a powder-type auxiliary odor control agent;
(f) about 0.001 wt. % to 15 wt. % for a soluble metal salt-type auxiliary odor control agent; and (g) about 0.001 wt. % to about 80 wt. % of one or more active agents that are release agents selected from bioactive agent, surface active agent, medicinally and/or hygienically active agent, cosmetically active agent, or stimulating agents such as, nicotine and its salts, caffeine, cannabinoids; essential oil; flavoring agent; or sweetening agent; wherein the wt. % is based on the total activated composition weight, and with the proviso that the sum of the percentages of (a) to (g) equals 100 percent.

19. The composition of claim 1, which comprises:
(a) about 5 wt. % to about 99.99 wt. % of spherical activated carbon particles comprising a sphericity of at least 0.6, a particle diameter in the range of about 0.01 μm to about 5000 μm, a BET-specific surface area of from 100 to 2500 m2/g, or further an iodine adsorption capacity of 500 mg/g or greater;
(b) about 0.01 wt. % to about 10 wt. % of spherical activated carbon particles comprising water;
(c) about 0.1 wt. % to about 80 wt. % of a treatment compound of an acid, base, or urea, or mixtures thereof,
(d) about 0.01 wt. % to about 15 wt. % of a colloidal particle;
(e) about 0.01 wt. % to about 80 wt. % of a powder-type auxiliary odor control agent;
(f) about 0.001 wt. % to about 5 wt. % for a soluble metal salt-type auxiliary odor control agent;
(g) about 0.01 wt. % to about 25 wt. % of one or more active agents selected from the group of bioactive agents, essential oils, flavoring agents, sweetening agents, surface active agents, medicinally and/or hygienically active agents, cosmetically active agents, or stimulating agents such as nicotine and its salts, caffeine and its salts, and/or cannabinoids oil and its salts; and
(h) about 0.01 wt. % to about 80 wt. % of phase change material (PCM); wherein the wt. % is based on the total activated composition weight and
with the proviso that the sum of the percentages of (a) to (h) equals 100 percent.

20. A use of the composition of claim 1, wherein the composition is an effective malodor control agent for any undesired aroma, complex fishy smells containing no amines, sulfur-containing malodors, and complex sulfur-containing malodors (represented by Kimchi malodors), including volatile acid malodors, volatile aldehydes, including formaldehyde, or VOC (volatile organic compounds) such as ethylene gases.

21. An article in consumer, personal care, home care, and healthcare-related products and industrial applications comprising an activated composition of claim 1 from about 0.01 wt. % to about 99.99 wt. % based on the total weight of the article, wherein the article effectively sequesters one or more of: complex fishy smells derived from marine omega-3 oil; complex sulfur-containing compounds derived from Kimchi; volatile organic aldehydes, including formaldehyde; volatile organic acids; or volatile organic compounds including ethylene.
